(19) United States
(12) Patent Application Publication
(10) Pub. No.: US 2007/0027362 A1
(43) Pub. Date: Feb. 1, 2007

(54) Infrared observation system

(75) Inventors: Keiji Handa, Tokyo (JP); Kenji Harano, Tokyo (JP); Hiroyuki Nishida, Sagamihara-shi (JP); Hiroshi Ishiwata, Tokyo (JP); Daisuke Asada, Tokyo (JP)

Correspondence Address: Thomas Spinelli;Scully, Scott, Murphy & Presser, Suite 300, 400 Garden City Plaza, Garden City, NY, 11530, US

(73) Assignee: OLYMPUS MEDICAL SYSTEMS CORP., TOKYO (JP)

(21) Appl. No.: 11/494,136

(22) Filed: Jul. 27, 2006

(30) Foreign Application Priority Data
Jul. 27, 2005 (JP) 2005-217682
Sep. 14, 2005 (JP) 2005-267388
Sep. 15, 2005 (JP) 2005-269021

(51) Int. Cl. A61B 1/06

(52) U.S. Cl. 600/160

(57) ABSTRACT

An infrared observation system comprises: a light source unit for generating illumination light for irradiating light including infrared light of a long wavelength exceeding at least a 1000-nm wavelength upon a living body tissue inside or outside the body in a broadband or a narrowband; an infrared image capturing unit for capturing an image using infrared light of a wavelength band exceeding 1000 nm in the light reflected from or transmitted through at the living body tissue; and an identifying unit for facilitating identification between a case in which living body tissue is blood or a blood vessel, and a case in which the living body tissue is other living body tissue, using the difference of moisture extinction properties in the wavelength band exceeding a wavelength of 1000 nm.

This application claims benefit of Japanese Application Nos. 2005-217682 filed on Jul. 27, 2005, 2005-267388 filed on Sep. 14, 2005, and 2005-269021 filed on Sep. 15, 2005, the contents of which are incorporated by this reference.

### BACKGROUND OF THE INVENTION

1. Field of the Invention

The present invention relates to an infrared observation system adapted for observing a blood vessel or the like at the deep portion side of a living body.

2. Description of the Related Art

Heretofore, as for technology for identifying the course of a blood vessel, a method for observing the course of a blood vessel using hemoglobin extinction properties of a near-infrared wavelength of 700 nm through 1000 nm has been available.

For example, as for a first preceding example, Japanese Unexamined Patent Application Publication No. 2004-358051 has disclosed technology in which the properties of in-blood hemoglobin that absorbs infrared light are used to obtain an image of a blood vessel of living body tissue, which cannot readily be observed by visible light, by using infrared light as illumination light.

FIG. 2 in this patent document illustrates photo-absorption properties of hemoglobin within a vein and oxygenated hemoglobin within an artery. Observation of a vein and an artery of a surface layer is facilitated by modifying a filter employed for observation according to the difference of these properties.

Also, as for a second preceding example, WO 2002/075289 has disclosed a device and a method for measuring a hematocrit value using an emission optical apparatus and a photon detection optical apparatus of a wavelength within a range of 800 nm through 1000 nm, and a wavelength within a range of 1250 nm through 1600 nm.

### SUMMARY OF THE INVENTION

An infrared observation system according to the present invention comprises a light source unit for generating illumination light for irradiating light including infrared light of a long wavelength exceeding at least a 1000-nm wavelength upon a living body tissue inside or outside the body in a broadband or a narrowband, an infrared image capturing unit for capturing an image using infrared light of a wavelength band exceeding 1000 nm in the light reflected from or transmitted through the living body tissue, and an identifying unit for facilitating identification between the case in which a living body tissue is blood or a blood vessel and the case of other living body tissue using the difference of moisture extinction properties in the wavelength band exceeding 1000 nm.

### BRIEF DESCRIPTION OF THE DRAWINGS

FIG. 1 is a block diagram illustrating the overall configuration of an infrared observation system according to a first embodiment of the present invention;

FIG. 2 is a properties diagram illustrating water transmittance properties;

FIG. 3 is a properties diagram illustrating transmittance properties such as a blood vessel, muscle, and so forth making up a living body;

FIG. 4 is a schematic action explanatory diagram according to the present embodiment;

FIG. 5 is a block diagram illustrating the configuration of a CCU;

FIG. 6 is a block diagram illustrating the configuration of the enhancement-level determining circuit shown in FIG. 5;

FIG. 7 is a properties diagram illustrating one example of the conversion properties of the enhancement-level conversion unit shown in FIG. 5;

FIG. 8 is a block diagram illustrating the configuration of a weighting-coefficient determining circuit shown in FIG. 5;

FIG. 9 is a properties diagram illustrating one example of the coefficient properties of the weighting-coefficient creation unit shown in FIG. 8;

FIG. 10 is a configuration diagram illustrating the configuration of the enhancement processing circuit shown in FIG. 5;

FIG. 11 is an explanatory diagram illustrating operation of the image processing device shown in FIG. 5;

FIG. 12 is a block diagram illustrating the configuration of a CCU according to a first modification;

FIG. 13 is a properties diagram illustrating the enhancement-level conversion properties of the enhancement-level conversion unit shown in FIG. 12;

FIG. 14 is a configuration diagram illustrating the configuration of an enhancement processing circuit;

FIG. 15 is a block diagram illustrating the configuration of a CCU according to a second modification;

FIG. 16 is a block diagram illustrating the overall configuration of an infrared observation system according to a second embodiment of the present invention;

FIG. 17 is a block diagram illustrating the overall configuration of the infrared observation system according to a modification of the second embodiment;

FIG. 18 is a block diagram illustrating the overall configuration of an infrared observation system according to a third embodiment of the present invention;

FIG. 19 is a block diagram illustrating the overall configuration according to a modification of the third embodiment;

FIG. 20 is a block diagram illustrating the overall configuration of an infrared observation system according to a fourth embodiment of the present invention;

FIG. 21 is a diagram illustrating the internal configuration of a capsule-type endoscope;

FIG. 22 is a diagram illustrating the internal configuration of a capsule-type endoscope according to a modification of the fourth embodiment;

FIG. 23 is a block diagram illustrating the overall configuration of an infrared microscope system according to a fifth embodiment of the present invention;

FIG. 24 is a diagram illustrating the overall configuration of an infrared observation system according to a sixth embodiment of the present invention;

FIG. 25 is a conceptual action explanatory diagram of a situation in which a blood vessel is observed by irradiating infrared light upon a living body tissue serving as a subject;

FIG. 26 is a diagram illustrating the transmittance properties of a blood vessel and fat in a living body tissue.

FIG. 27 is a diagram illustrating a schematic image example obtained in a case of observing a blood vessel covered with fat using visible region light;

FIG. 28 is a diagram illustrating a schematic image example obtained using the infrared observation system according to the sixth embodiment;

FIG. 29 is an overall configuration diagram according to a first modification of the sixth embodiment;

FIG. 30 is an overall configuration diagram according to a second modification of the sixth embodiment;

FIG. 31 is an overall configuration diagram of an infrared observation system according to a seventh embodiment;

FIG. 32 is an overall configuration of an infrared observation system according to a modification of the seventh embodiment;

FIG. 33 is a diagram illustrating one example of the configuration of principal portions of an infrared observation system according to an eighth embodiment;

FIG. 34 is a diagram illustrating one example of the relation between the wavelength band and light transmittance of light to be irradiated at fat and the tube wall of a blood vessel.

FIG. 35 is a diagram illustrating one example of the placement state of a light source device and an image capturing device at the time of capturing the image of a blood vessel using the infrared observation system according to the eighth embodiment;

FIG. 36 is a diagram illustrating one example of an image in which a blood vessel course to be displayed on a monitor at the time of capturing the image of a blood vessel using the infrared observation system according to the eighth embodiment is visualized;

FIG. 37 is a diagram illustrating one example of the relation between the wavelength band and radiance of light to be irradiated in a halogen lamp;

FIG. 38 is a diagram illustrating the placement state of a light source device and an image capturing device at the time of capturing the image of a blood vessel using the infrared observation system according to the eighth embodiment;

FIG. 39 is a diagram illustrating one example of the relation between the wavelength band and light transmittance of light to be irradiated at fat, the tube wall of a blood vessel, and blood;

FIG. 40 is a diagram illustrating one example of the configuration of principal portions of an infrared observation system according to a ninth embodiment;

FIG. 41 is a diagram illustrating one example of the relation between the wavelength band and light transmittance of light to be irradiated at fat, the tube wall of a blood vessel, and blood;

FIG. 42 is a diagram illustrating the placement state of a light source device and an image capturing device at the time of obtaining a blood vessel course state using the infrared observation system according to the ninth embodiment;

FIG. 43 is a diagram illustrating one example of an image in which a blood vessel course to be displayed on a monitor at the time of capturing the image of a blood vessel using the infrared observation system according to the ninth embodiment is visualized;

FIG. 44 is a diagram illustrating one example different from FIG. 19 of the placement state of a light source device and an image capturing device at the time of obtaining a blood vessel course state using the infrared observation system according to the ninth embodiment;

FIG. 45 is a diagram illustrating the configuration of principal portions of an endoscope system according to a tenth embodiment of the present invention;

FIG. 46 is a diagram illustrating one example of the configuration of treatment equipment to be employed for performing observation using an endoscope system;

FIG. 47 is a diagram illustrating the photo-absorption properties of hemoglobin and oxygenated hemoglobin;

FIG. 48 is a diagram illustrating one example of the placement state of the endoscope and the treatment equipment in the case of capturing the image of a subject using the endoscope constituting the endoscope system according to the tenth embodiment and the treatment equipment shown in FIG. 46;

FIG. 49 is a diagram illustrating another configuration example of treatment equipment to be employed at the time of performing observation using the endoscope system according to the tenth embodiment;

FIG. 50 is a diagram illustrating one example of the configuration of a fiber provided in the treatment equipment shown in FIG. 49;

FIG. 51 is a diagram illustrating another configuration example of treatment equipment to be employed at the time of performing observation using the endoscope system according to the tenth embodiment;

FIG. 52 is a diagram illustrating one example of the state in which the multiple surface members provided in the treatment equipment shown in FIG. 51 are each moved rotationally in a predetermined direction;

FIG. 53 is a diagram illustrating one example of the configuration of one surface member of the multiple surface members provided in the treatment equipment shown in FIG. 51;

FIG. 54 is a diagram illustrating a configuration example different from the surface member shown in FIG. 53, of the multiple surface members provided in the treatment equipment shown in FIG. 51;

FIG. 55 is a diagram illustrating one example of the configuration of a shaft member for attaching the surface members shown in FIGS. 53 and 54; and

FIG. 56 is a diagram illustrating one example of the placement state of the endoscope and the fiber cable in the case of capturing the image of a subject using an endoscope constituting the endoscope system according to the tenth embodiment and a fiber cable in which LEDs for emitting infrared light are provided.

### DESCRIPTION OF THE PREFERRED EMBODIMENTS

Hereinafter, embodiments of the present invention will be described with reference to the drawings.

### First Embodiment

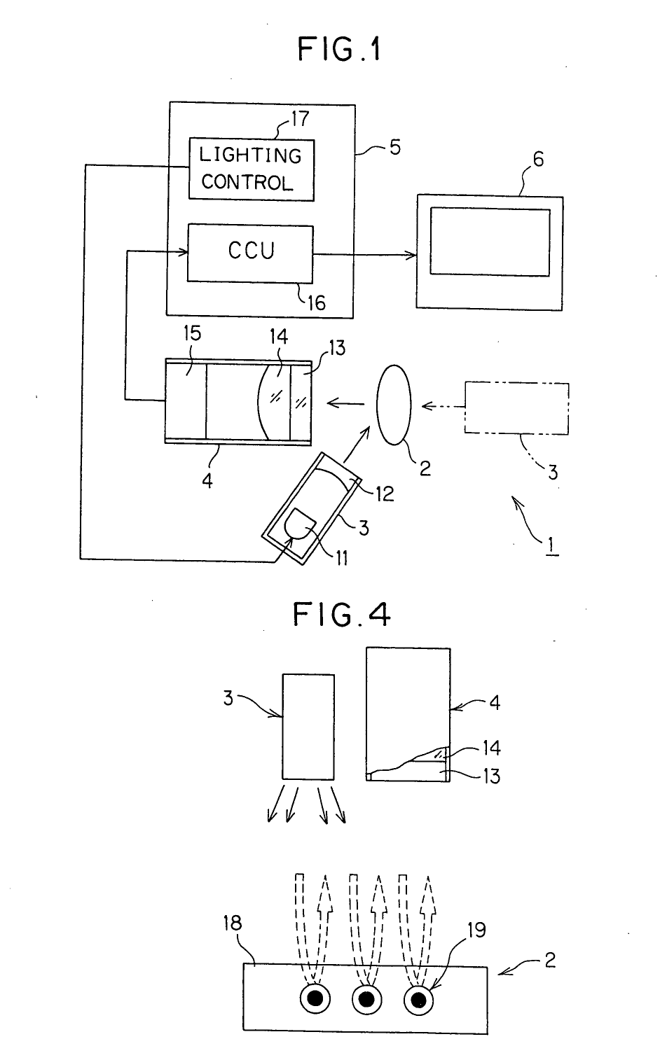

A first embodiment of the present invention will be described with reference to FIGS. 1 through 15.

As illustrated in FIG. 1, an infrared observation system 1 according to the first embodiment of the present invention comprises a light source device 3 for irradiating light including infrared light as illumination light upon, for example, a living body 2 serving as an object to be observed, an infrared image capturing camera 4 serving as infrared image capturing means (hereinafter, simply abbreviated as infrared camera) for performing image capturing using the infrared light in the light reflected at the living body 2 at which this illumination light is irradiated, or in the light transmitted such as illustrated in a two-dot chain line, a control device 5 for subjecting the image capturing signal captured by the infrared camera 4 to signal processing, and a monitor 6 for displaying a picture signal to be output from the control device 5.

The light source device 3 incorporates a lamp 11 such as a halogen lamp, tungsten lamp, or the like, for generating illumination light with the range from a visual region (band) to an infrared band having a long wavelength exceeding at least a wavelength of 1000 nm.

The lamp 11 is preferably a lamp having great emission intensity in an infrared wavelength band to be employed for later described image capturing. As for the lamp 11, a halogen lamp having continuous emission properties up to a wavelength band exceeding 3000 nm can be employed, for example.

The illumination light obtained by turning on the lamp 11 is irradiated upon the living body 2 through an illumination lens 12. The image based on the reflection light at the time of irradiating this illumination light upon the living body 2 is formed on the image capturing surface of an image capturing device 15 through a filter 13 and an image-formation lens 14 constituting the infrared camera 4 serving as image capturing means having infrared sensitivity exceeding at least a wavelength of 1000 nm.

Note that FIG. 1 illustrates that the infrared camera 4 is configured so as to receive the reflection light from the living body 2 at the time of irradiating the illumination light at the living body 2 from the light source device 3, but the light source device 3 may be disposed such as illustrated in a two-dot chain line for example. Subsequently, the infrared camera 4 may be the infrared observation system 1 configured so as to receive the transmission light by the living body 2.

The image capturing device 15 employed for the above infrared camera 4 is an image capturing device constituted of a semiconductor detecting device (photovoltaic semiconductor detecting element), for example, such as Ex. InGaAs, InAs, InSb, or the like, having sensitivity in an infrared wavelength band exceeding at least a 1000-nm wavelength. These image capturing devices have sensitivity in a wavelength band at least from 1000 nm to 2550 nm or so. Note that InAS and InSb have sensitivity even as to light having a wavelength equal to or longer than 3000 nm which is longer than 2550 nm.

Also, the wavelength band which the filter 13 disposed at the front of the image capturing device 15 transmits is set such as described in the following.

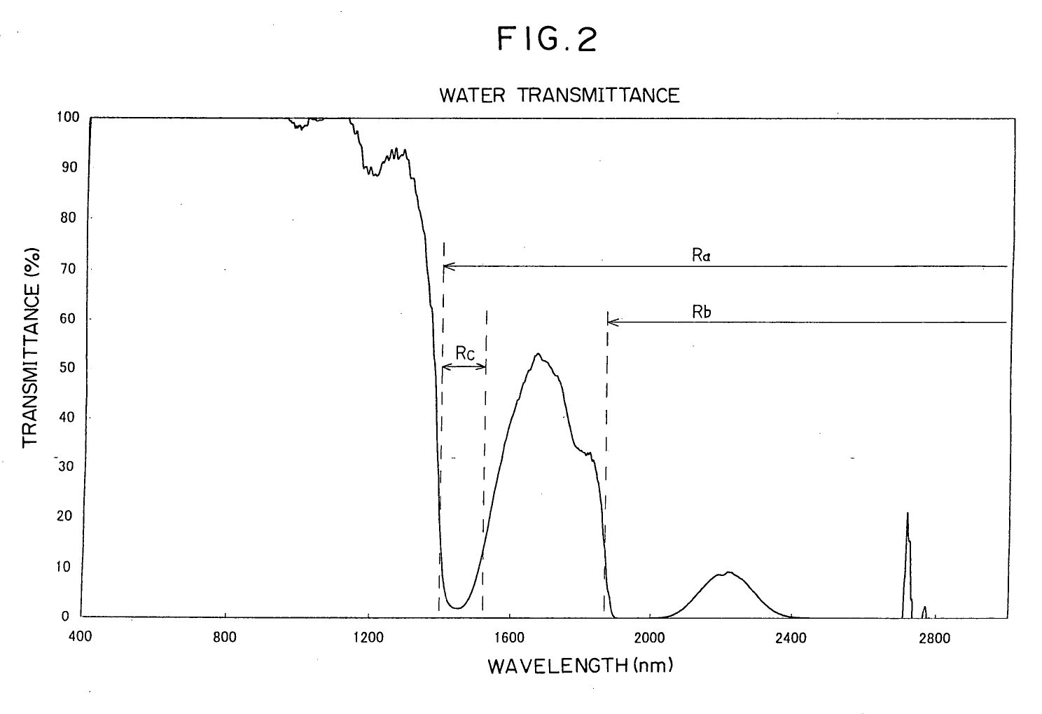

As illustrated in FIG. 2, this wavelength band is set to a wavelength band which facilitates identification between a case in which the tissue included in the living body 2 in near-infrared light exceeding a 1000-nm wavelength is blood or a blood vessel and a case in which the tissue is other living body tissue including cases of a fat tissue and an organ.

As illustrated in FIG. 2, water exhibits high transmission properties in a visible wavelength being almost never absorbed, and exhibits high transmission properties even longer wavelength side than this, but exhibits the properties in which the transmittance suddenly decreases to around 0% in a wavelength of around 1400 nm, and subsequently, increases again in a wavelength of around 1500 nm, reaches around 50%, following which returns to 0% again in a wavelength of around 1900 nm.

Also, the tissues and organs of the living body 2 have moisture content such as shown in the following list.

| TISSUES AND ORGANS | MOISTURE CONTENT |
|---|---|
| MUSCLE | 76% |
| LIVER | 68% |
| FAT TISSUE | 10% |
| BLOOD | 91% |

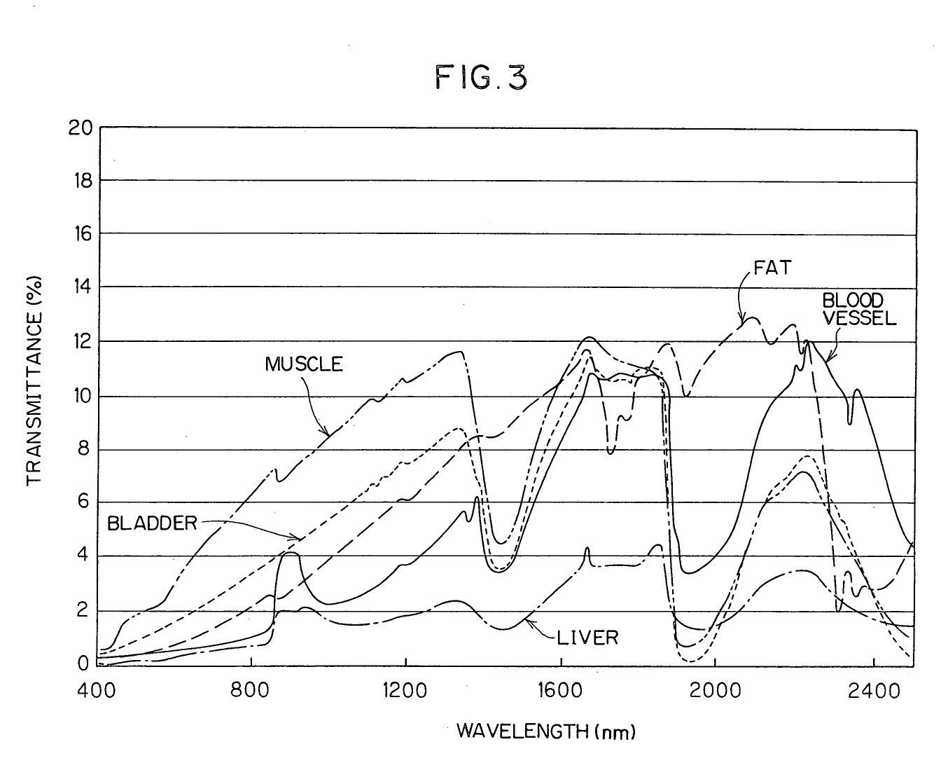

Also, FIG. 3 schematically illustrates the properties of the measurement results of transmittance in each case of the muscles, fat, blood vessels, bladder, and liver of the living body 2.

The blood vessels are made up of generally the same tissue as the muscles, but blood within the blood vessels include water close to 90% as can be understood from the above list, so blood greatly differs from fat including water of 10% or so regarding moisture content. Also, blood differs regarding moisture content around 20% from the other organs which are living body tissue, for example, the liver having moisture content of 70%.

Consequently, by performing image capturing using moisture extinction properties in a specific wavelength band in which the moisture extinction properties are characteristic properties, the captured image information is expected to serve as image information representing moisture content of the specific wavelength band.

Accordingly, for example, with water transmittance properties as illustrated in FIG. 2, the transmission wavelength of the filter 13 is set such that a broadband wavelength Ra equal to or longer than a wavelength of 1400 nm in which water transmittance rapidly varies, and the value of the transmittance decreases (i.e., absorptivity increases), or a broadband wavelength Rb equal to or longer than a wavelength of 1900 nm, or a narrowband or an inter-band wavelength Rc of 1400 nm through 1500 nm, serves as a specific wavelength band employed for image capturing.

Note that these specific wavelength bands Ra, Rb, and Rc may be set to further narrow part of a wavelength band (broadband, inter- or narrowband). In this case, these specific wavelength bands Ra, Rb, and Rc may be set in light of the transmittance properties (absorptivity properties) of the other living body tissues. Also, infrared image capturing means for performing infrared image capturing may be constituted of the light of separated multiple wavelength bands.

Making of such settings constitutes identifying means which facilitates identification of the difference present regarding water transmittance in a case in which the living body tissue in an portion to be observed is blood, having properties quite close to those of moisture, and in a case in which the living body tissue is fat or the like having less moisture content than in former case.

In other words, with the image obtained by image capturing, an arrangement is made such that a portion having a low illumination level principally corresponds to a blood portion, and inversely, a portion having a high illumination level principally corresponds to the other living body tissue portion including less moisture, such as fat or the like.

Thus, with the present embodiment, the above identifying means is formed by the setting of the transmission properties of a specific wavelength area by the filter 13.

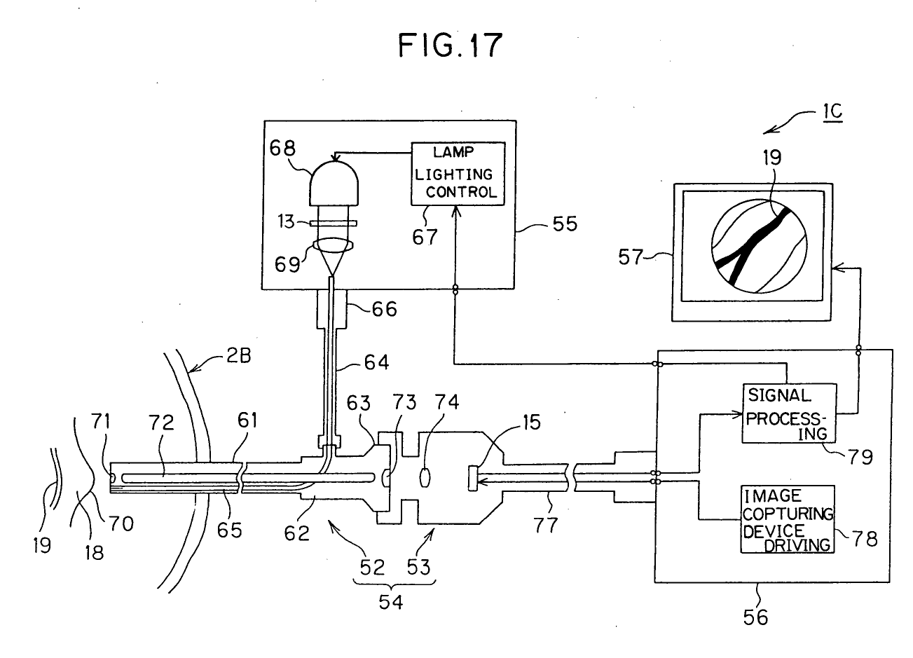

A specific example of operation according to the present embodiment will be described later with reference to FIG. 4. FIG. 4 schematically illustrates the case of observing a state in which a blood vessel 19 is running underneath of the living body 2 and is covered by fat tissue 18. Note that later-described FIG. 16 illustrates this state more specifically.

Illumination light from visible light to infrared light is irradiated upon the living body 2 from the light source device 3, but the optical image to be formed at the image capturing device 15 constituting infrared image capturing means is formed with light having a specific wavelength which is transmitted through the filter 13.

That is to say, with the present embodiment, the light image-captured by the image capturing device 15 is set so as to perform image capturing with a wavelength band in which absorptivity by moisture at a longer wavelength side than a wavelength of 1000 nm is sufficiently small. In addition, this specific wavelength band is a wavelength band exhibiting quite high transmittance as to a fat tissue having a little moisture content.

Accordingly, the light components of the illumination light emitted from the light source device 3 which contribute to actual image capturing by the image capturing device 15 can be arranged so as to be transmitted through the tissue of the fat 18 with relatively small attenuation, and reach the deep portion side of the living body tissue.

Subsequently, an arrangement is made wherein a readily identifiable image can be obtained such that the difference of absorptivity between blood and the fat 18 accompanies great illumination level difference within the image to be captured by the image capturing device 15.

Thus, with the present embodiment, an arrangement is made wherein the filter 13 disposed in front of the image capturing device 15 of the infrared camera 4 employs, by using the difference of moisture extinction properties, a wavelength band which facilitates identification between blood or blood vessel 19 and the other living body tissues including a case of the fat 18 or the other organs as the wavelength band of image capturing light to be employed for actual image capturing by the image capturing device 15.

Note that in FIG. 4, description will be made regarding the case of blood, and regarding the case of fat 18 which includes a very little moisture content as the living body tissue other than blood, but the other organs such as the liver and the like have in-between properties between the two as an overall tendency, so that moisture extinction properties can also be effectively employed for identifying between blood or blood vessels and organs as other living body tissue.

The optical image formed on the image capturing surface of the image capturing device 15 is subjected to photoelectric conversion by the image capturing device 15. The image capturing device. 15 outputs the signal subjected to photoelectric conversion as an image capturing signal by the driving signal being applied to the image capturing device 15 from an unshown driving circuit within a camera control unit (abbreviated as CCU) 16 built in the control device 5. This image capturing signal is input to the CCU 16, and is converted into a picture signal by an unshown picture signal generating circuit within the CCU 16.

Subsequently, this picture signal is output to the monitor 6, and the display screen of the monitor 6 displays the image captured by the image capturing device 15. Also, the control device 5 lights and drives a lamp 11 within the light source device 3, and also incorporates a lighting control circuit 17 which enables the amount of emission thereof to be controlled.

The infrared observation system 1 according to the present embodiment having such a configuration has actions such as illustrated in the schematic diagram in FIG. 4. Note that FIG. 4 illustrates a case of infrared observation in reflection light.

As illustrated in FIG. 4, the illumination light which covers the range from visible light to infrared light is irradiated upon the living body 2 from the light source device 3. Subsequently, the reflection light from the living body 2 is image-captured by the infrared camera 4. With the living body 2, the blood vessel 19 is often in a state covered with the tissue of the fat 18.

Consequently, the amount of attenuation becomes great in infrared light at a shorter wavelength side than a wavelength of 1000 nm or so as well as the case of visible illumination light, it is difficult to capture an image with the reflection light from the blood vessel 19 at the underside of the fat 18.

Alternatively, with the present embodiment, the image capturing device 15 having sensitivity at a longer wavelength side than a wavelength of 1000 nm is employed, and also illumination light including a longer wavelength side than a wavelength of 1000 nm is irradiated as illumination light. Also, with a wavelength band wherein transmittance properties as to water having almost the same transmittance properties (extinction properties in other words) as the blood flowing inside the blood vessel 19 are extremely reduced, the filter 13 to which the transmission wavelength band thereof is set is disposed in front of the image capturing device 15.

Alternatively, for example, the fat 18 including a little moisture content has transmittance decreased at a longer wavelength side than 2300 nm such as illustrated in FIG. 3 in the passage Ra or Rb which the filter 13 lets through, but has great transmittance at the shorter wavelength side than that. Also, even with the case of the passage Rc which the filter 13 lets through, though the passage band thereof is narrowed, this has basically the same tendency (features).

Accordingly, with the wavelength employed for image capturing of the image capturing device 15, a state in which the transmittance as to the tissue of the fat 18 is high is maintained, and the illumination light reaches the tissue of the blood vessel 19 with little attenuation. Subsequently, this is greatly absorbed by the blood within the blood vessel 19, so that the intensity greatly differs between the reflection light from the blood within the blood vessel 19 and the reflection light from the surrounding tissues thereof, such as the fat 18 and so forth (becomes reflection light). That is to say, as illustrated with a dotted line in FIG. 4, the reflection light upon which the course state of the blood vessel 19 covering the blood is reflected is obtained.

Accordingly, in the event that the image capturing signal to be output from the image capturing device 15 for receiving reflection light and capturing an image is subjected to signal processing by the CCU 16 to generate a picture signal and display this on the monitor, an image having contrast which greatly differs between blood or blood vessel 19 and the tissue of the fat 18 can be obtained.

With the above description, setting the image capturing light to be cast into the image capturing device 15 so as to reflect water characteristic transmittance properties thereupon can provide the image information to be obtained by the image capturing device 15 for capturing an image based on the reflection light or transmission light from the living body 2 upon which the identification of a living body tissue is reflected using the difference of water extinction properties.

Alternatively, performing the image processing (signal processing) further corresponding to water characteristic transmittance properties as described below at the CCU 16 side for subjecting the image capturing signal captured by the image capturing device 15 to signal processing (image processing) may constitute identifying means for facilitating identification of a living body tissue using the difference of water extinction properties.

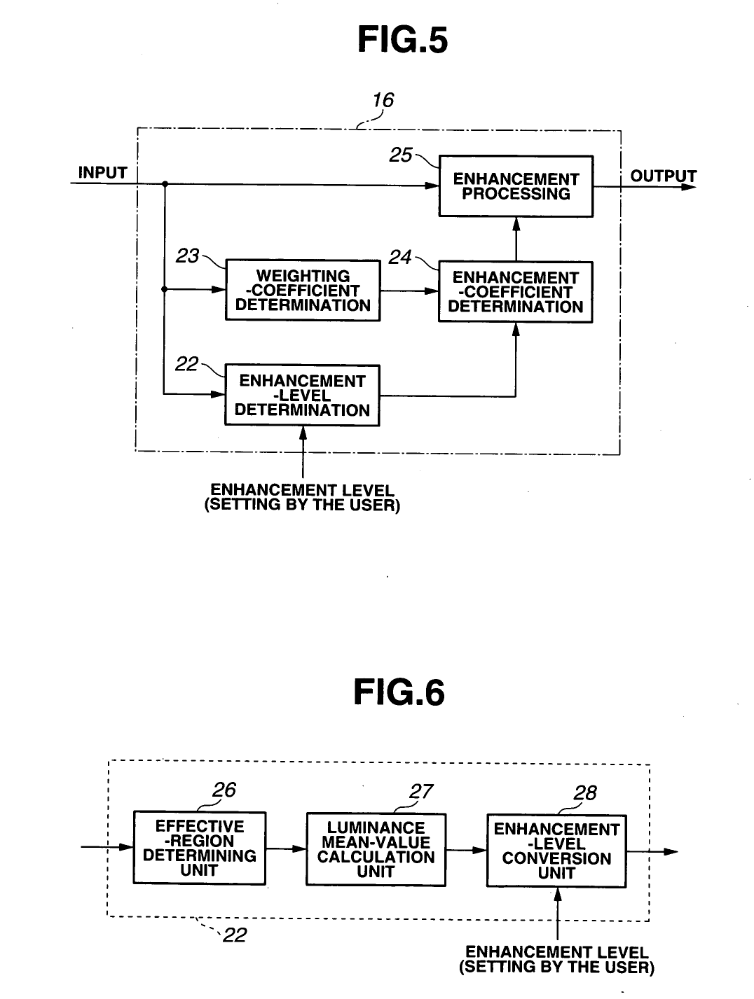

FIGS. 5 through 11 relate to image processing according to the present embodiment.

As illustrated in FIG. 5, the CCU 16 according to the present embodiment is an image processing device for performing contrast enhancement as to an image signal to be input from the image capturing device 15, and includes an enhancement-level determining circuit 22 serving as amount-of-features computing means for determining an enhancement level for each captured image based on the mean luminance value of the effective regions of the image capturing signal and the enhancement level set by a user.

Also, the CCU 16 includes a weighting-coefficient determining circuit 23 for weighting the amount of enhancement for each pixel based on the luminance value of an image, an enhancement-coefficient determining circuit 24 serving as enhancement level setting means for determining the enhancement coefficient for each pixel based on the output from the above enhancement-level determining circuit 22 and the above weighting-coefficient determining circuit 23, and an enhancement processing circuit 25 serving as enhancement processing means for performing enhancement processing as to the image of the image capturing signal based on the enhancement coefficient determined by the enhancement-coefficient determining circuit 24.

As illustrated in FIG. 6, the enhancement-level determining circuit 22 comprises an effective-region determining unit 26 for extracting an effective region from the image of an image capturing signal to be input, a luminance mean-value calculation unit 27 for calculating the mean value of the luminance values within an effective region, and an enhancement-level conversion unit 28 for determining the enhancement level for each image based on the enhancement level and the luminance mean value set by the user.

The enhancement-level determining circuit 22 first extracts a region, excluding around halation portions and around dark portions of an image, to be input by the effective-region determining unit 26, as an effective region. Extraction of an effective region is performed with reference to the luminance value for each pixel. For example, in the event that an image to be input has a 8-bit accuracy, 230 or higher on the 256-grayscale is taken as being around a halation portion, and 50 or lower is taken as being around a dark portion, and the enhancement-level determining circuit 22 extracts the region which is not included in these portions as an effective region.

Next, the luminance mean-value calculation unit 27 calculates the mean value of the luminance values of a pixel determined as an effective region, and the-enhancement-level conversion unit 28 determines the enhancement level for each image based on the luminance mean value calculated by the luminance mean-value calculation unit 27 and the enhancement level set by the user.

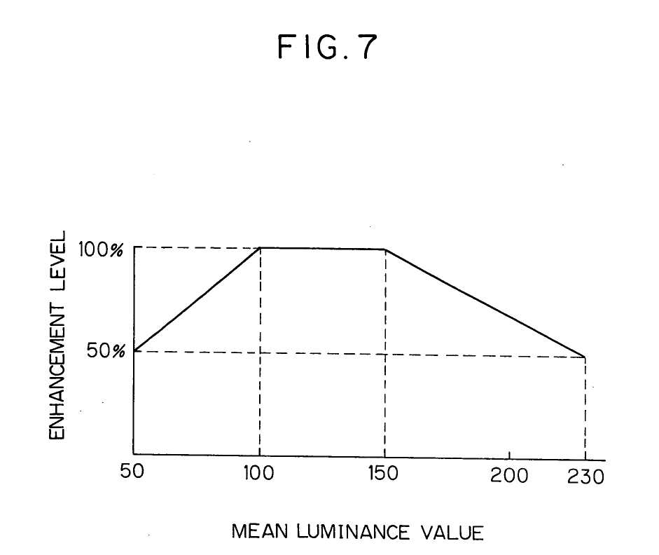

FIG. 7 is a diagram illustrating one example of the conversion properties of the enhancement-level conversion unit 28. The enhancement level set by the user is converted based on a luminance mean value to be input. The enhancement level set by the user is output as it is when the luminance mean value is in a range from 100 to 150 on the 256-grayscale, and the value from 100% to 50% of the enhancement level set by the user is output when the image has another luminance mean value.

Thus, the enhancement-level determining circuit 22 is configured so as to suppress enhancement as to the region under observation which is an extremely bright image or an extremely dark image which needs little enhancement, with reference to the mean luminance of the effective region of the image.

In other words, as described above, the tissue portion of fat and blood can be identified with relatively great contrast, but identification can be further facilitated by performing such image enhancement as to a portion exhibiting in-between properties of the two (e.g., in the case of a blood portion and a liver portion).

Note that identification can be further facilitated by performing such image processing as to the case of the tissue portion of fat and blood. Also, even as to a case in which the wavelength band of the image capturing light is not set to such a specific wavelength band, identifying means for facilitating identification by enhancing the contrast difference at the image processing side may be configured.

Also, even in the event that the image capturing light is set to a specific wavelength band such as described above, in order to further facilitate identification of a blood vessel course at a further deep portion side of the living body, enhancement processing may be performed using the conversion properties or the like such as illustrated in FIG. 7. Also, the conversion properties may be set to the properties different from the properties illustrated in FIG. 7.

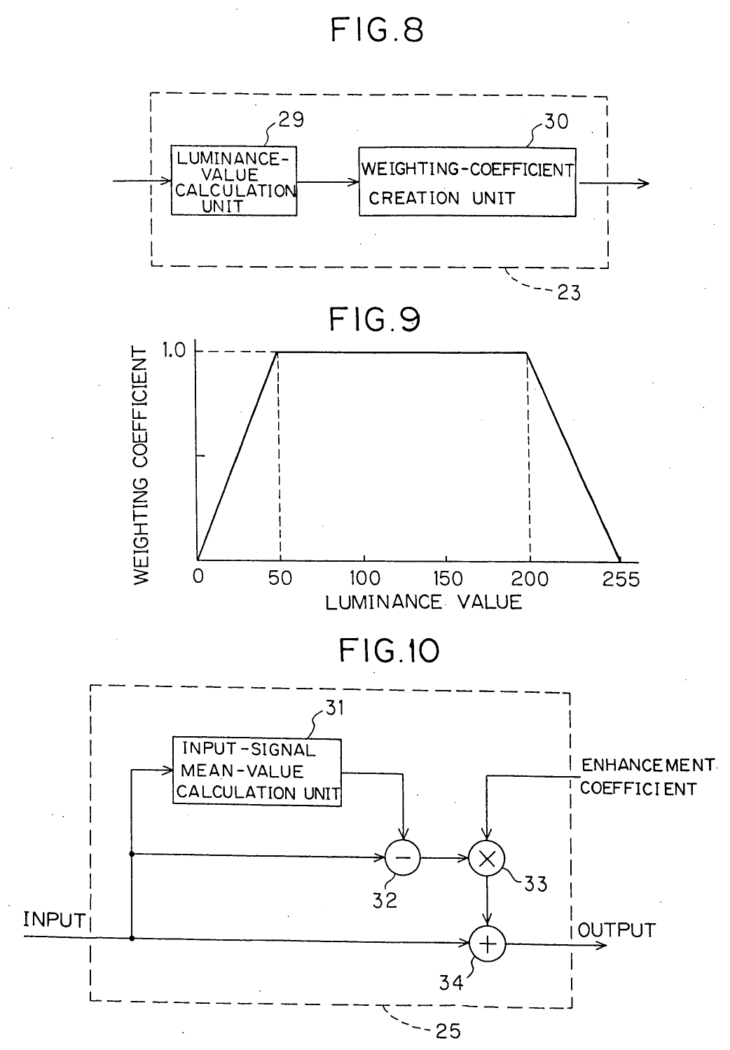

Also, as illustrated in FIG. 8, the weighting-coefficient determining circuit 23 comprises a luminance-value calculation unit 29 for calculating the luminance value for each pixel from an image (of an image capturing signal) to be input, and a weighting-coefficient creation unit 30 for creating a weighting coefficient for performing weighting as to an enhancement coefficient with reference to a luminance value.

The luminance-value calculation unit 29 calculates the luminance value for each pixel of an image to be input. The calculated luminance value is input to the weighting-coefficient creation unit 30, and is converted using the properties such as illustrated in FIG. 9 for example to create a weighting coefficient. That is to say, the weighting-coefficient creation unit 30 outputs 1.0 as a weighting coefficient when the luminance value is in a range from 50 to 200 on the 256-grayscale, and outputs a value from 0 to 1.0 when the luminance value has the other value.

Accordingly, the weighting-coefficient determining circuit 23 is configured so as to perform operation for suppressing enhancement as to a region unsuitable for enhancement, such as a halation perimeter portion, a dark perimeter portion, and so forth within an image. Also, the weighting-coefficient determining circuit 23 is configured so as not to perform enhancement as to a portion of which enhancement is unnecessary.

Subsequently, the enhancement-coefficient determining circuit 24 multiplies the enhancement level output from the enhancement-level determining circuit 22 by the weighting coefficient output from the weighting-coefficient determining circuit 23 to determine the enhancement coefficient for each pixel. The enhancement-coefficient determining circuit 24 determines an enhancement coefficient based on the enhancement level set by the conditions of the entire image, and the weighting coefficient set by the conditions for each pixel.

As illustrated in FIG. 10, the enhancement processing circuit 25 comprises an input-signal mean-value calculation unit 31 for calculating the mean value of image capturing signals to be input, a subtracter 32 for performing subtraction between an input signal and an input signal mean value, a multiplier 33 for multiplying an enhancement coefficient by the above subtracted value, and an adder 34 for adding the above input signal mean value and the output from the above multiplier 33.

The input-signal mean-value calculation unit 31 calculates the mean value of image signals (image capturing signals) to be input which is the center of enhancement, and the subtracter 32 subtracts the above mean value from image signals to be input. The subtracted value is multiplied by the enhancement coefficient output from the enhancement-coefficient determining circuit 24 at the multiplier 33, and the difference from the above mean value is enhanced. The difference between the enhanced input image signal and the above mean value is added with the above mean value by the adder 34. That is to say, the enhancement processing circuit 25 in calculate the following Expression (1).
|o=(|i−|a)×α+|a (1)
wherein |i represents an image signal (image capturing signal) to be input, |o represents an image signal to be output, |a represents the mean value of image signals to be input, and α represents an enhancement coefficient.

The CCU 16 according to the present embodiment thus configured first determines, based on the image of an image capturing signal to be input at the enhancement-level determining circuit 22, the enhancement level of the entire image. The determination of the enhancement level is performed with reference to the enhancement level set by the user, the histogram of the mean luminance value or the luminance values of the effective regions of an image, the amount of features obtained by an image to be input, and so forth.

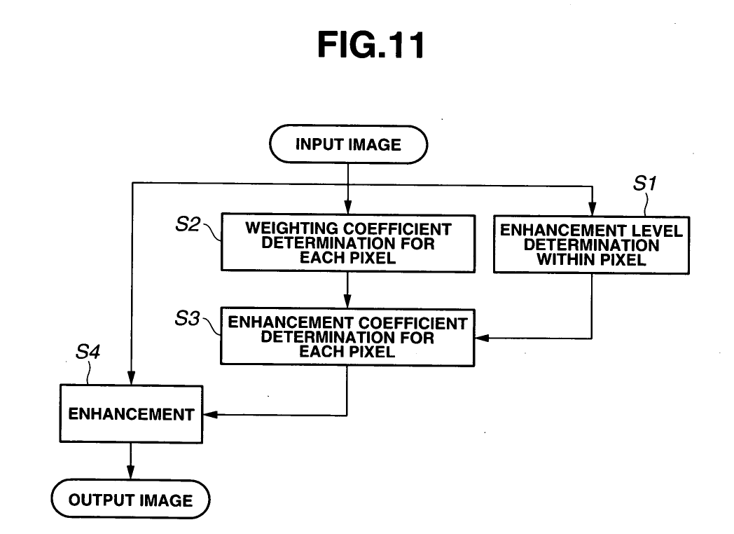

For example, upon performing enhancement as it is in the event that the mean luminance value of an image is markedly high, and also the enhancement level set by the user is great, many bright regions included cause the regions, which bring about overexposure, to be outstanding, resulting in an image which cannot be observed easily in some cases. Accordingly, as illustrated in FIG. 11, in step S1, the enhancement-level determining circuit 22 automatically sets again an enhancement level lower than the enhancement level set by the user.

Also, the weighting-coefficient determining circuit 23 determines a weighting coefficient to perform weighting of the enhancement coefficient for each pixel from an image to be input. This is performed to prevent change in the original image from becoming unrecognizable when setting an enhancement level to strong, such as around a halation portion, around a dark portion, and so forth within the image. Here, as with step S1, in step S2, weighting is performed except for regions around a halation portion and around a dark portion with reference to the luminance value for each pixel and so forth.

Next, in step S3, the enhancement-coefficient determining circuit 24 determines the enhancement coefficient for each pixel based on the enhancement level and the weighting coefficient set in steps S1 and S2. For example, the enhancement coefficient for each pixel is determined by multiplying the enhancement level determined in step S1 by the weighting coefficient determined in step S2.

Subsequently, in step S4, the enhancement processing circuit 25 subjects to enhancement an image for input, based on the enhancement coefficient determined for each pixel.

The above flow realizes enhancement processing with a different enhancement level for each image, an enhancement image which can be readily observed can be obtained even from a markedly bright image or dark image which needs no enhancement, around a halation portion, around a dark portion, and so forth without setting an enhancement level again.

That is to say, with the CCU 16 according to the present embodiment, the enhancement coefficient of a halation perimeter portion or a dark perimeter portion which readily causes color information loss by being enhanced is automatically set low, so the region under observation can be principally enhanced without decreasing the enhancement level.

Even regarding a portion having a small difference of moisture content, an image which further facilitates identification can be displayed by enhancing the difference of the moisture content. For example, between a liver portion and a blood portion has a little difference of moisture content as compared with between a fat portion and a blood portion, so the difference of contrast of the two becomes small, but an image which has more contrast and facilitates identification can be provided by performing enhancement processing.

Also, even in the event that the mean luminance value of an effective region except for around a halation portion and around a dark portion is markedly high, or markedly low, the enhancement level of the entire image can be automatically set low, whereby an enhanced image which can be readily observed can be obtained without setting an enhancement level again.

Also, the following arrangement may be made as a first modification of the CCU 116.

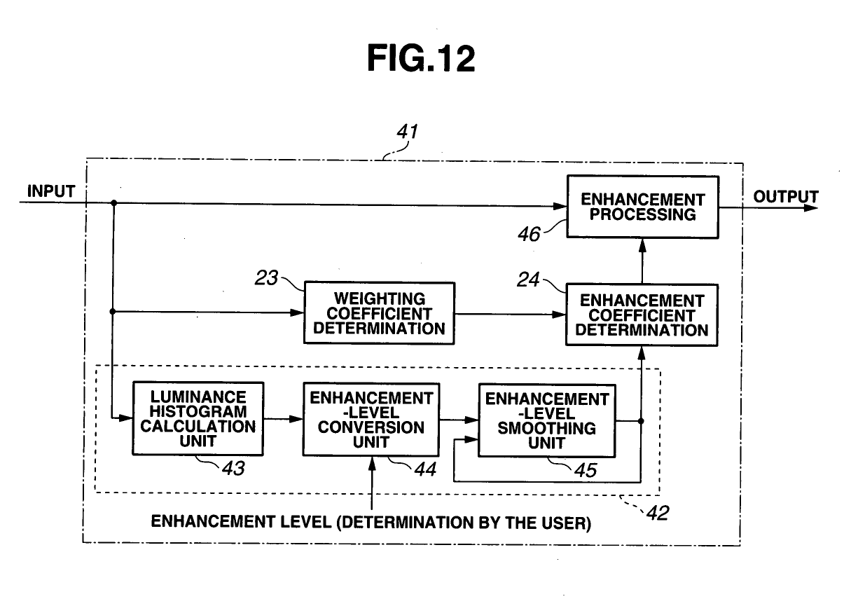

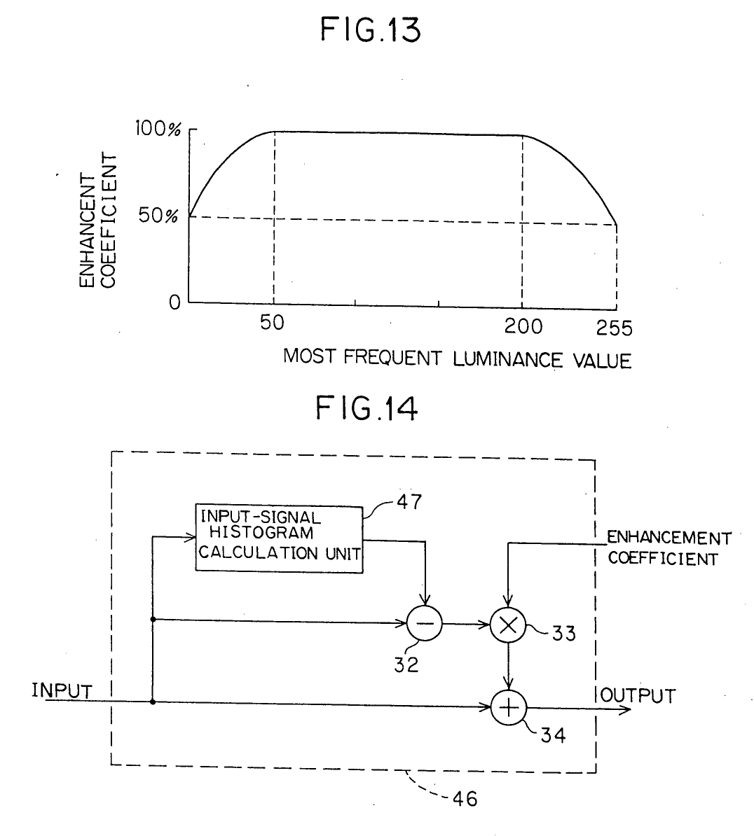

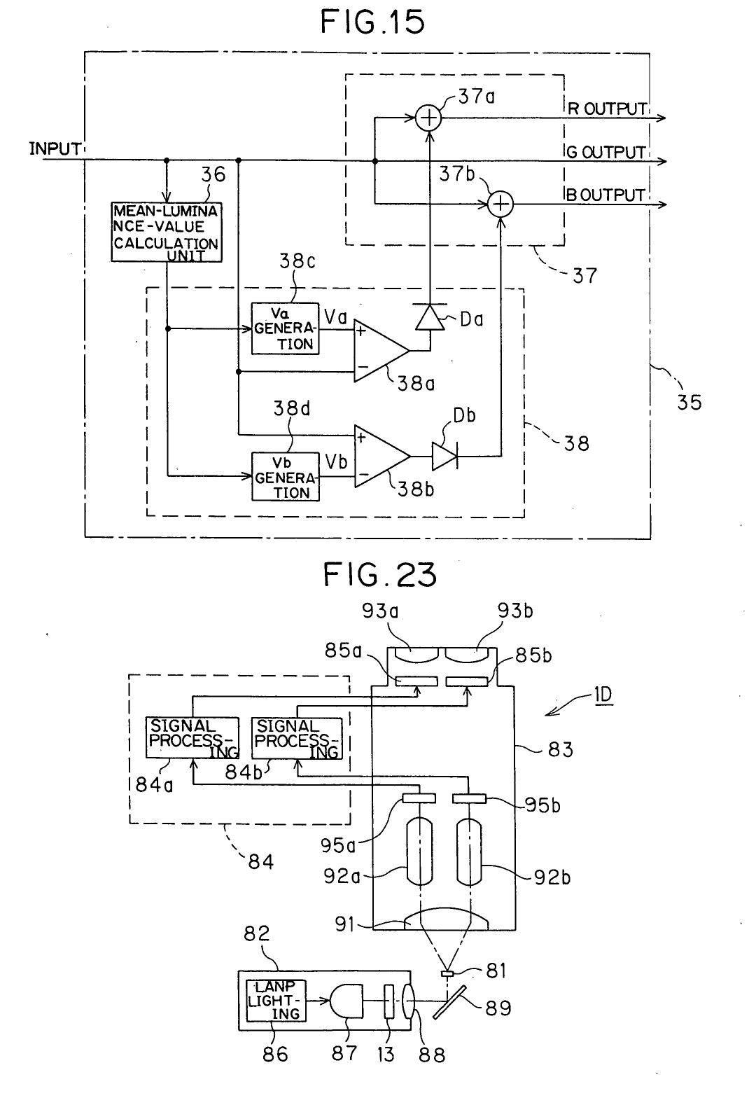

FIGS. 12 through 14 relate to the first modification of the CCU 16, wherein FIG. 12 is a configuration diagram illustrating the configuration of the CCU, FIG. 13 is a properties diagram illustrating the enhancement-level conversion properties of the enhancement-level conversion unit shown in FIG. 12, and FIG. 14 is a configuration diagram illustrating the configuration of the enhancement processing circuit in FIG. 12.

The present modification has almost the same configuration as the case of the first embodiment, so only the different points will be described, and the same configurations will be denoted with the same reference numerals, and description thereof will be omitted.

With the present modification, the weighting-coefficient determining circuit 23 and the enhancement-coefficient determining circuit 24 have the same internal configuration as those in the first embodiment (see FIG. 5), and the other enhancement-level determining circuit 42 and enhancement processing circuit 46 have a different configuration from those in the first embodiment.

That is to say, instead of the luminance mean value, which is taken as the index of weighting in the first embodiment, a weighting coefficient is determined using the most frequent value obtained from the histogram of luminance.

Specifically, as illustrated in FIG. 12, the enhancement-level determining circuit 42 in a CCU 41 according to the present modification comprises a luminance histogram calculation unit 43, an enhancement-level conversion unit 44, and an enhancement-level smoothing unit 45.

With the enhancement-level determining circuit 42, the luminance histogram calculation unit 43 calculates the luminance histogram of an image to be input, detects the highest frequent luminance value from the calculated histogram, and outputs this to the enhancement-level conversion unit 44 on the subsequent stage.

The enhancement-level conversion unit 44 performs, based on the enhancement level set by the user and the highest frequent luminance value calculated by the above luminance-histogram calculation unit 43, conversion of the enhancement level.

As with the first embodiment, as illustrated in FIG. 13 for example, in the event that an image to be input is 8 bits, the enhancement-level conversion unit 44 outputs the enhancement level set by the user as it is when detecting the most frequent luminance value is in a range from 50 to 200 of 256-grayscale.

In the event of detecting the other most frequent luminance value, the enhancement-level conversion unit 44 outputs the value of 50% through 100% of the enhancement level set by the user. Note that with the present modification, in the event of detecting the most frequent luminance value equal to or less than 50, or equal to or greater than 200, let us say that conversion is performed using the properties having a quadratic function.

The enhancement level output from the enhancement-level conversion unit 44 is input to the enhancement-level smoothing unit 45. The enhancement-level smoothing unit 45 subjects the enhancement level which changes for each image to temporal smoothing using a recursive filter or the like, and suppresses rapid change in an enhancement level to be generated in the event that movement of a subject is rapid, and so forth.

The enhancement processing circuit 46 is, as illustrated in FIG. 14, configured so as to calculate the center of enhancement by an input-signal histogram calculation unit 47 instead of the input-signal mean-value calculation unit 31 in the first embodiment. The input-signal histogram calculation unit 47 calculates the histogram of an input signal, and detects the most frequent input signal value.

The detected most frequent input signal value is input to the subtracter 32 on the subsequent stage, where the difference as to the input signal is calculated. The subsequent processing has the same configuration and operations as that in the first embodiment, where contrast enhancement centered on the most frequent value of an input signal is performed.

Accordingly, even with the present modification, an image processing device which can obtain the same advantages as with the first embodiment can be realized. Also, with the present modification, a recursive filter is employed for determination of an enhancement level, the enhancement level is subjected to smoothing in the temporal direction, and rapid change in an enhancement level to be generated in the event that movement of a subject is rapid, and so forth is suppressed, and accordingly, the present modification adapts to a case in which contrast enhancement processing is performed using moving images. Note that with the present modification, an enhancement level is subjected to smoothing in the temporal direction, but smoothing may be performed using a spatial filter with reference to the enhancement levels of surrounding pixels.

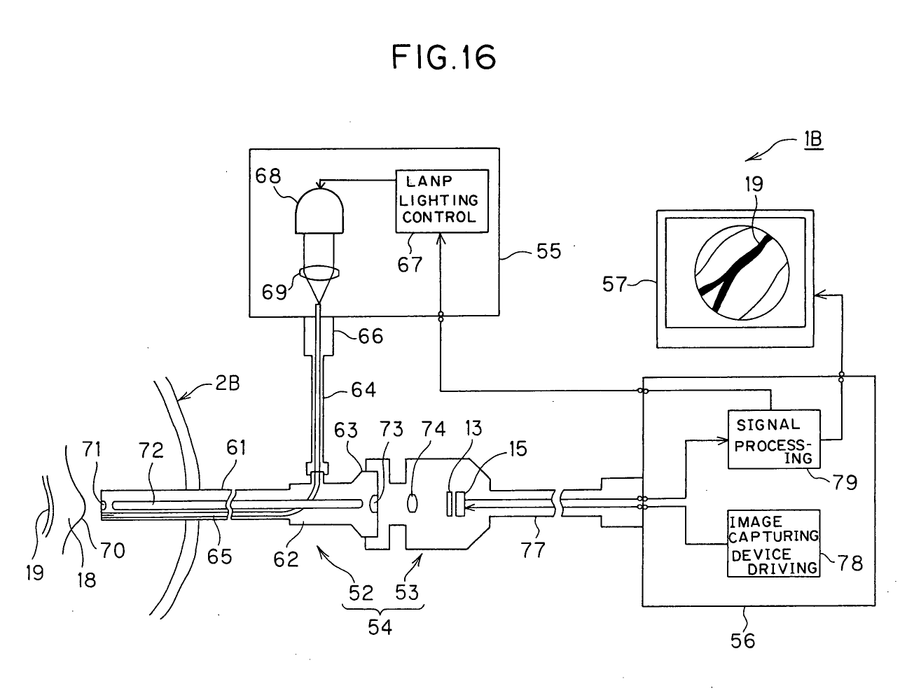

Also, an arrangement may be made wherein with a configuration such as a CCU 35 according to a second modification illustrated in FIG. 15, an easily identifiable image is displayed by performing image processing for converting a color tone depending on a luminance level. With the second modification, for example, image processing for displaying a portion having a low luminance level and a portion having a high luminance level with a different color tone is performed respectively. Here, description will be made with reference to a simple example, but processing such as excluding an extremely bright portion and an extremely dark portion as described above may be performed.

The CCU 35 includes a mean-luminance-value calculation unit 36 for calculating the mean luminance value of an image capturing signal to be input, an RGB-signal generating unit 37 for generating color signals from an image capturing signal to be input, e.g., RGB signals, and outputting these to the monitor 6, and an enhancement signal generating unit 38 for generating a signal to be subjected to color enhancement in accordance with the luminance level of an image capturing signal.

The RGB-signal generating unit 37 outputs, from an image capturing signal input, a G signal as it is, and an R signal and a B signal which are obtained by adding the image capturing signal at adders 37a and 37b respectively. Accordingly, when a signal to be input to the adders 37a and 37b from the enhancement signal generating unit 38 side is 0, monochrome RGB signals are output to the monitor 6.

Also, the image capturing signal is input to, for example, two subtracters 38a and 38b constituting the enhancement signal generating unit 38, and two enhancement signals are generated.

The enhancement signal generating unit 38 generates, for example, a first enhancement signal which is lower than a first threshold value Va lower than the mean luminance value in the luminance level of the image capturing signal, and a second enhancement signal higher than a second threshold value Vb higher than the mean luminance value.

Subsequently, the RGB-signal generating unit 37 is configured so as to perform enhancement processing for enhancing a red color tone using the first enhancement signal, and inversely, perform enhancement processing for enhancing a blue color tone using the second enhancement signal higher than the mean luminance value.

Accordingly, the subtracter 38a, for example, outputs the value obtained by subtracting the image capturing signal from the first threshold value Va generated by a first threshold-value generating unit 38c (e.g., using the mean luminance value) to the adder 37a constituting the RGB-signal generating unit 37 via a diode Da. The adder 37a adds the image capturing signal and the output signal of the subtracter 38a, and outputs this result as an R signal.

Note that the first threshold-value generating unit 38c performs scaling using the mean luminance value, and generates the first threshold value Va which is set lower than the mean luminance value. For example, when assuming that the mean luminance value is <V>, Va=a·<V> holds. Here, a is restricted to 0<a<1. More specifically, the first threshold value Va enhances and displays such a low luminance level portion so as to become a red color tone to facilitate identification of a portion with luminance level close to blood.

Accordingly, upon a signal having a low luminance level like as blood being input to the subtracter 38a, this signal is subtracted from the first threshold value Va, and the luminance of the R signal is increased in accordance with the level of the difference signal thereof.

Also, the other subtracter 38b outputs the value obtained by subtracting the second threshold value Vb generated by a second threshold-value generating unit 38d (e.g., using the mean luminance value) from the image capturing signal to the adder 37b constituting the RGB-signal generating unit 37 via a diode Db.

The adder 37b adds the image capturing signal and the output signal of the subtracter 38b, and outputs this result as a B signal. Note that the second threshold-value generating unit 38d performs scaling using the mean luminance value, and generates the second threshold value Vb which is set higher than the mean luminance value.

For example, when assuming that the mean luminance value is <V>, Vb=b<V> holds, wherein b is restricted to 1<b. More specifically, the second threshold value Vb enhances and displays such a high luminance level portion so as to become a blue color tone to facilitate identification of a living body tissue having a little moisture, more specifically, a portion with luminance level close to the tissue of fat.

Accordingly, upon a signal having a high luminance level like fat being input to the subtracter 38b, the second threshold value Vb is subtracted from this signal, and the luminance of the B signal is increased in accordance with the level of the difference signal thereof.

Accordingly, the user can recognize the portion of the blood 19 where blood is running and the tissue portion of the fat 18 in a more identifiable state based on the color tone of the image to be displayed on the monitor.

Note that an arrangement may be made wherein the number of the subtracters 38a and 38b constituting the enhancement-signal generating unit 38 is increased, even a portion where the difference of luminance levels is small is subjected to color enhancement as described above, thereby generating a color image which further facilitates identification.

Also, an arrangement may be made wherein a user is allowed to change and set the values of the first threshold value Va and second threshold value Vb, and around the luminance level corresponding to the selection or setting of the user is subjected to color enhancement and displayed.

Thus, according to the present embodiment, the image capturing light employed for image capturing is set to a specific wavelength band including a wavelength band where moisture extinction properties are characteristic according to a living body tissue, or the image captured is subjected to image processing using the difference of moisture absorption properties, whereby image information which facilitates identification between the case of the blood or blood vessel and the case of the other living body tissues including the case of fat tissue or the other organ can be obtained.

### Second Embodiment

Next, a second embodiment of the present invention will be described with reference to FIGS. 16 and 17. FIG. 16 illustrates an infrared observation system according to the second embodiment of the present invention.

As illustrated in FIG. 16, an infrared observation system 1B according to the second embodiment of the present invention comprises a camera mounting endoscope (hereinafter, simply abbreviated as scope) 54 mounting, for example, a camera head 53 which incorporates image capturing means within an optical endoscope 52 to be inserted in the abdomen 2B (of a living body 2), a light source device 55 for supplying illumination light to the optical endoscope 52, a CCU 56 for performing signal processing as to the image capturing means built in the camera head 53, and a monitor 57 for displaying the endoscope image captured by the image capturing means with the standard picture signal output from the CCU 56 being input.

The optical endoscope 52 includes, for example, a hard insertion portion 61, a gripper 62 provided at the back end of the insertion portion 61, and an ocular portion 63 provided at the back end of the gripper 62, and the mouthpiece of the gripper 62 is connected to a light guide cable 64.

A light guide 65 for transmitting illumination light is inserted within the insertion portion 61, and with the light guide 65, a light guide connector 66 provided at the end portion thereof is detachably connected to the light source device 55 via the light guide cable 64 connected to the mouthpiece of the side portion of the gripper 62.

A lamp 68 such as a halogen lamp or the like which is turned on by lamp lighting power source to be supplied from a lamp lighting control circuit 67 is provided within the light source device 55, and the lamp 68 generates from visible light to infrared light far exceeding a wavelength of 1000 nm as described above.

The light of the lamp 68 is condensed at a condenser lens 69 disposed on an illumination light path, illumination light is cast into the incident end surface of the light guide 65 of the light guide connector 66, and is transmitted to the tip surface (emitting end surface) of the insertion portion 61 by the light guide 65.

Subsequently, the illumination light is emitted from the tip surface of the light guide 65, and is emitted toward an observation object portion 70 side such as stomach or the like within the abdomen 2B, and illuminates the observation object portion 70.

An objective lens 71 is attached to an observation window provided adjacent to an illumination window at the tip portion of the insertion portion 61, and forms an optical image of the observation object portion 70 such as an illuminated affected portion or the like. The optical image is transmitted to the back end surface side by a relay lens system 72 serving as an image guide.

The transmitted optical image can be enlarged and observed using an ocular lens 73 provided at the ocular portion 63. In the event that the camera head 53 is mounted on the ocular portion 63, the transmitted optical image is formed at the image capturing device 15 via an image capturing lens 74 within the camera head 53.

In this case, for example, the filter 13 which has been described in the first embodiment is disposed within the optical path between the image capturing lens 74 and the image capturing device 15.

Also, a camera cable 77 extending from the camera head 53 is connected to the CCU 56. The CCU 56 comprises an image capturing device driving circuit 78 and a signal processing circuit 79, and the image capturing device driving circuit 78 applies an image capturing device driving signal to the image capturing device 15.

Subsequently, the image capturing signal subjected to photoelectric conversion by the image capturing device 15 to which the image capturing device driving signal is applied is input in the signal processing circuit 79. The signal processing circuit 79 subjects the input image capturing signal to signal processing for generating a picture signal.

Subsequently, the generated picture signal is output to a monitor 57, and the image captured by the image capturing device 15 is displayed on the display screen of the monitor 57.

Also, a dimming signal representing the mean brightness in several-frames period thereof as to the luminance level of the picture signal in the signal processing circuit 79 is input to the lamp lighting control circuit 67 within the light source device 55. Subsequently, the lamp lighting control circuit 67 controls the amount of emission of the lamp 68 by the difference signal between the dimming signal and an unshown reference brightness signal.

Next, with the infrared observation system 1B thus configured, surgery performed upon the stomach to be treated within the abdomen 2B under observation using the scope 54 will be described.

With the infrared observation system 1B according to the present embodiment, for example, in order to cut open the stomach to be treated, covered with an omentum majus or the like, which has been cancerated or the like, by inserting the insertion portion 61 of the scope 54 into the inside of the abdomen 2B via an unshown trocar as illustrated in FIG. 16, it is sometimes necessary to confirm the course of the blood vessel 19.

In this case, the omentum majus portion is adhered with the tissue of the fat 18 in the case of an adult or the like, the tissue causes the omentum majus portion to become thick, and consequently, as described above, the tissue of the fat 18 makes it difficult for the user to confirm using visible light or near-infrared light the course of the blood vessel 19 in which the blood is flowing.

Alternatively, in the event that the blood vessel 19 is running on the underside (inside) covered with the observation object portion 70 made up of the tissue of the fat 18 such as an omentum majus or the like as illustrated in FIG. 16, with the present embodiment, by an image capturing using light having a specific wavelength band exceeding a wavelength of 1000 nm (illumination light itself is a broader band), an image which allows the user to recognize the course of the blood vessel 19 portion where the blood is flowing can be obtained, such as schematically illustrated on the monitor 57 in FIG. 16 as with the description in the first embodiment.

That is to say, according to the present embodiment, photo-absorption is characteristically performed in a blood portion exhibiting almost the same extinction properties as moisture, so that an image wherein the blood vessel 19 where blood is flowing has a lower luminance level than the tissue portion of the fat 18, i.e., a contrast-enhanced image which facilitates the user to recognize blood vessel course can be obtained wherein contrast becomes dark in the blood vessel 19 portion, and contrast becomes bright in the fat 18 tissue.

Thus, even in the event that an endoscope is inserted within a body cavity to perform surgery under the endoscope, the present embodiment allows the surgeon to recognize the course of the blood vessel 19 under the fat 18 or the like, and facilitates rapid treatment while suppressing bleeding. Accordingly, the time for surgery can be greatly reduced, whereby both burden of a surgeon and burden of a patient can be greatly reduced.

FIG. 17 illustrates an infrared observation system 1C according to a modification of the second embodiment. The infrared observation system 1C is configured wherein the filter 13 disposed within the camera head 53 in the infrared observation system 1B in FIG. 16 is moved into the light source device 55.

That is to say, of the light by the lamp 68, only light of a specific wavelength band in infrared light is transmitted by the filter 13, and is irradiated at the observation object portion 70 side via the light guide 65. Subsequently, the reflection light from the observation object portion 70 side is received by the image capturing device 15. The other configurations are the same configuration as the infrared observation system 1B in FIG. 16.

The actions and advantages of the present modification are almost the same as those in the case of the infrared observation system 1B in FIG. 16. Thus, according to the present embodiment and the present modification, even in the event that surgery within a body cavity under endoscope observation or the like is performed, identification between the case of blood or blood vessel and the case of the other living body tissues including the case of fat tissue or an organ can be facilitated.

Accordingly, surgery as to the inside of a body cavity can be performed in a short period of time and also in a smooth manner. Both burden of a surgeon and burden of a patient can be greatly reduced. Note that even with the present embodiment, an arrangement may be made wherein the image processing described in the first embodiment is performed.

### Third Embodiment

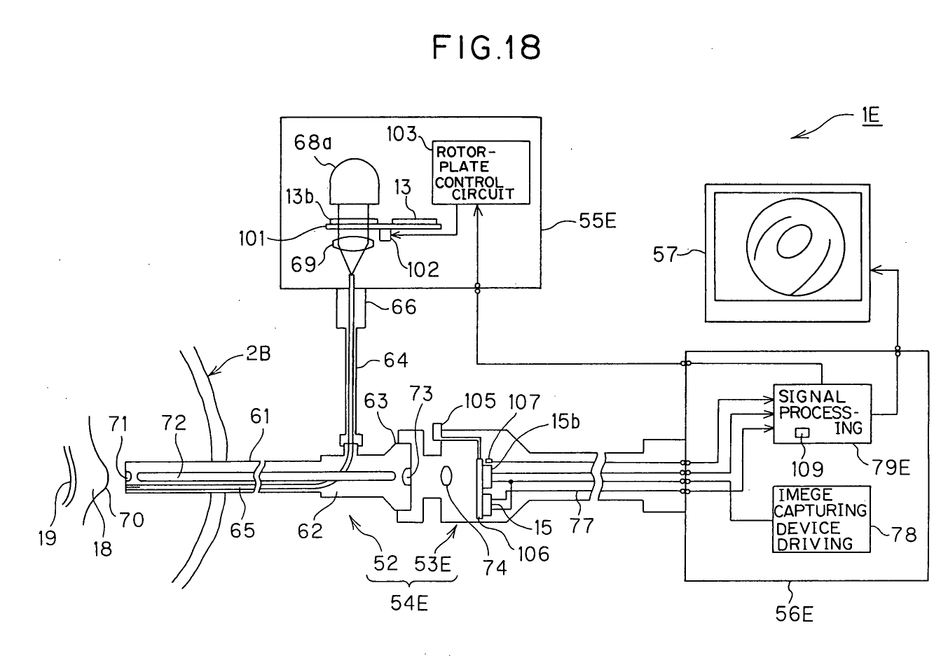

Next, an infrared observation system 1E according to a third embodiment of the present invention will be described with reference to FIG. 18. In addition to the infrared observation in the infrared observation system 1B according to the second embodiment, the infrared observation system 1E illustrated in FIG. 18 further enables visible observation to be performed. The configuration of the present embodiment is similar to the second embodiment, so the same components as the second embodiment are denoted with the same reference numerals, and description thereof will be omitted.

The infrared observation system 1E according to the present embodiment comprises a light source device 55E for selectively emitting infrared light and visible region light (abbreviated as visible light or ordinary light), a camera mounting endoscope (scope) 54E, a CCU 56E for performing signal processing as to the infrared image capturing device 15 and an ordinary light image capturing device 15b, which are provided in the camera head 53E constituting the scope 54E, and a monitor 57 for displaying the picture signal output from the CCU 56E.

With the light source device 55E, as the lamp 68 in the light source device 55 according to the second embodiment illustrated in FIG. 16, for example, a halogen lamp 68a for generating visible light and infrared light is employed, and also a rotor plate 101 is disposed in front of the halogen lamp 68a. With the rotor plate 101, the (infrared) filter 13 for transmitting the light of a specific wavelength band of infrared light, and an ordinary light filter 13b for transmitting ordinary light (visible light) alone are provided at two places facing each other in the circumferential direction.

The rotor plate 101 is rotated 180 degrees with a driving signal being applied from a rotor-plate control circuit 103 to a motor 102 attached to the center shaft thereof, thereby switching the filter to be disposed within an illumination light path.

That is to say, as illustrated in FIG. 18, in a state in which the ordinary light filter 13b is disposed within the optical path, ordinary light alone is emitted from the light source device 55E, and upon the ordinary light filter 13b being switched to the infrared light filter 13, the light having a specific wavelength band of infrared light is to be emitted.

Also, with the camera head 53E constituting the scope 54E according to the present embodiment, a switching plate 106 which provides a switching lever 105 is disposed in the camera head 53 constituting the scope 54 in FIG. 16, and the switching plate 106 has the infrared light image capturing device 15 and the ordinary light image capturing device 15b adjacently attached thereto.

Operating the switching lever 105 allows the surgeon to rotate the switching plate 106 by an appropriate angle, and selectively dispose the infrared light image capturing device 15 at a place where an image is formed via the image capturing lens 74, or selectively dispose the ordinary light image capturing device 15b. For example, FIG. 18 illustrates a state in which the ordinary light image capturing device 15b is disposed at an image-formation position, and the scope 54E is in an ordinary light image capturing state. In this state, upon the surgeon operating the switching lever 105, switching is performed such that the infrared light image capturing device 15 is disposed at an image-formation position.

As described above, the infrared light image capturing device 15 is an image capturing device having sensitivity in an infrared band employing InGaAs, InSb, or the like. On the other hand, the ordinary light image capturing device 15b is an image capturing device having sensitivity in a visible band, and is configured of a CCD or CMOS imager.

With these image capturing devices 15 and 15b, the signal connector of the end portion thereof is detachably connected to the CCU 56E via a signal line inserted into the inside of the camera cable 77.

The image capturing devices 15 and 15b each output a photoelectric converted image capturing signal with an image capturing driving signal applied to the image capturing devices 15 and 15b by the image capturing device driving circuit 78. FIG. 18 illustrates an example in which both of the image capturing devices 15 and 15b can be driven with a common driving signal for the sake of facilitating description, but these may be driven individually.

The image capturing signal to be output from the image capturing devices 15 and 15b is input to a signal processing circuit 79E, converted into a picture signal, and then output to the monitor 57, and the image captured by the image capturing device 15 or 15b is displayed on the display screen thereof.

Also, with the present embodiment, an arrangement is made wherein upon the image capturing state of the scope 54E being switched by operating the switching lever 105 as described below, the illumination state by the light source device 55E and the signal processing state by the CCU 56E can be switched in interlocking with the switching operation thereof.

Accordingly, an arrangement is made wherein in the event of operating the switching lever 105, the image capturing device which has been set in an image capturing state can be detected.

For example, upon the surgeon operating the switching lever 105 so as to rotate by a predetermined angle in the normal rotational direction or in the reverse rotational direction, in order to switch the image capturing device to be disposed at an image-formation position (from one to the other), position sensors 107 such as a photo reflector or the like provided at two places so as to face the switching plate 106 can detect the switching operation thereof, and also can detect the type of image capturing device set at the image-formation position.

Subsequently, the information of the position sensor 107 is input to the signal processing circuit 79E via the signal line within the camera cable. Subsequently, a CPU 109 serving as control means within the signal processing circuit 79E controls the signal processing in the signal processing circuit 79E so as to perform the signal processing corresponding to the image capturing device set at the image-formation position.

Also, the CPU 109 transmits information including detection of switching operation to the rotor-plate control circuit 103 within the light source device 55E via the signal line. Subsequently, when receiving the information, the rotor-plate control circuit 103 rotates the rotor plate 101 so as to emit illumination light corresponding to the image capturing state of the scope 54E.

Actions of the present embodiment using such a configuration will be described. FIG. 18 illustrates a state in which the scope 54E is inserted in the abdomen 2B, as described with reference to FIG. 16.

In this case, the insertion portion 61 of the scope 54E is inserted into the abdomen 2B via an unshown trocar. Subsequently, the observation object portion 70 such as an omentum majus or the like which covers the stomach therein is observed.

In such a case, the scope 54E is set to an ordinary light observation state by operating the switch lever 105, as illustrated in FIG. 18. In this state, the light source device 55E emits ordinary light, the ordinary light image capturing device 15b captures an image under the illumination of ordinary light, the CCU 56E further performs signal processing as to the ordinary light image capturing device 15b, and the monitor 57 color-displays the image captured by the ordinary light image capturing device 15b.

Thus, the surgeon can observe the inside of the abdomen 2B like by an ordinary endoscope. Specifically, the surgeon can observe the outline, shape, and so forth of the observation object portion 70 within the abdomen 2B, and can recognize whether or not it is the portion to be treated.

In this case, the observation of the outline and so forth of the surface of a living body tissue is performed, but in the event that the stomach to be treated is covered with the thick fat 18 tissue, it is necessary to recognize the course of the blood vessel 19 in the depth thereof and treat the omentum majus portion while reducing bleeding.

In such a case, the surgeon operates the switching lever 105 to dispose the infrared light image capturing device 15 at the image-formation position. The light source device 55E is in a state for emitting infrared light in interlocking with this switching operation, and also the CCU 56E is in a state for subjecting the infrared light image capturing device 15 to signal processing.

This state is the same state as the state described in the second embodiment. Subsequently, as described in the second embodiment, the surgeon can observe the course of the blood vessel 19. Consequently, enabling the course of the blood vessel 19 to be recognized enables the treatment to be performed in a smooth manner and in a short period of time. Thus, according to the present embodiment, illumination and image capturing (including signal processing) using infrared light and illumination and image capturing using ordinary light can be selected and set by the surgeon's switching operation, whereby one scope 54E can be employed for a wide range of applications, and observation and treatment can be performed in a smooth manner and in a short period of time.

In other words, when using an infrared light dedicated scope, observation using ordinary light cannot be performed, so it is necessary to spend time and effort such as exchanging the scope and so forth, but with the present embodiment, the scope 54E includes both functions, so can be used for both observations without spending time and effort for exchange, whereby excellent operability can be secured, and also observation or the like can be performed in a smooth manner and in a short period of time.

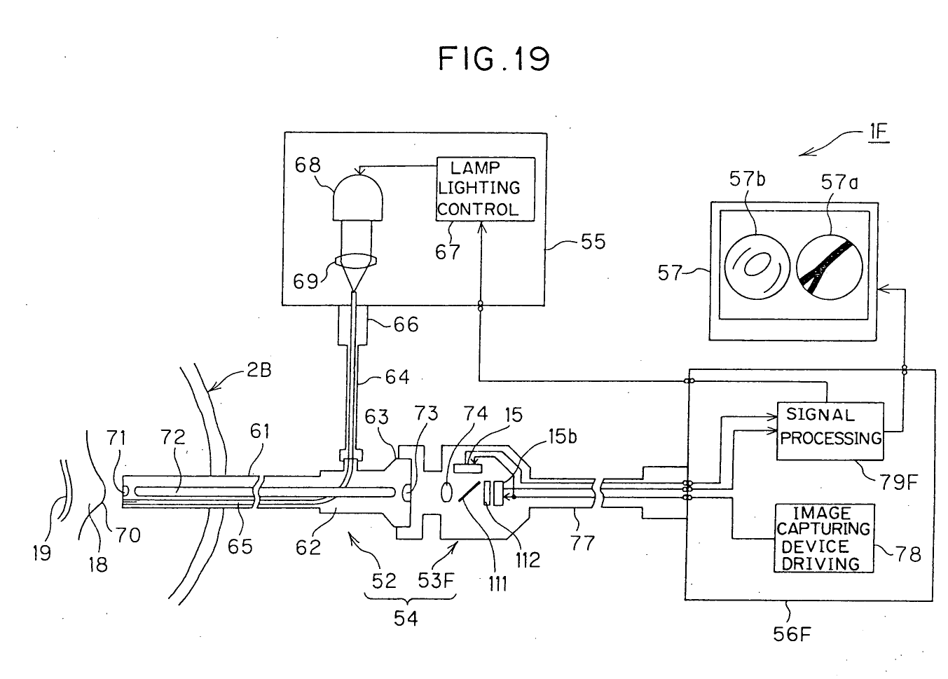

Next, a modification of the present embodiment will be described with reference to FIG. 19. With an infrared observation system 1F of the present modification, a dichroic mirror 111 is disposed within the camera head 53 in the infrared observation system 1B illustrated in FIG. 16. The image capturing device 15 is disposed at a position where an image based on the light reflected at the dichroic mirror 111 is formed, and the ordinary light image capturing device 15b is disposed at a position where an image based on the light which is transmitted through the dichroic mirror 111 is formed via an infrared cut filter 112.

The above dichroic mirror 111 selectively reflects a narrowband wavelength or in-between band wavelength Rc illustrated in FIG. 2, for example. Subsequently, image capturing is performed at the infrared light image capturing device 15 using the reflected light. On the other hand, the ordinary light image capturing device 15b performs image capturing using ordinary light (visible light).

These image capturing devices 15 and 15b are connected to a CCU 56F via a signal line. With the CCU 56F, the image capturing device driving circuit 78 drives both image capturing devices 15 and 15b simultaneously, the image capturing signals to be output from both image capturing devices 15 and 15b are input to a signal processing circuit 79F corresponding to two inputs, and are subjected to signal processing respectively, and a picture signal in which both images are mixed at an unshown mixing circuit (mixer) further inside thereof is generated. The display screen of the monitor 57 is configured so as to simultaneously display the images captured by both of the image capturing devices 15 and 15b.

FIG. 19 illustrates a situation wherein an infrared image 57a captured by the infrared light image capturing device 15 and an ordinary image 57b captured by the ordinary light image capturing device 15b are displayed simultaneously on the monitor 57.

According to the present modification, the infrared image 57a and the ordinary image 57b can be displayed without performing a switching operation. Accordingly, even the case of desiring to display both images simultaneously for comparison can be handled. Note that an arrangement may be made wherein one image is great, and the other is small, i.e., both images are displayed as parent-and-child images.

### Fourth Embodiment

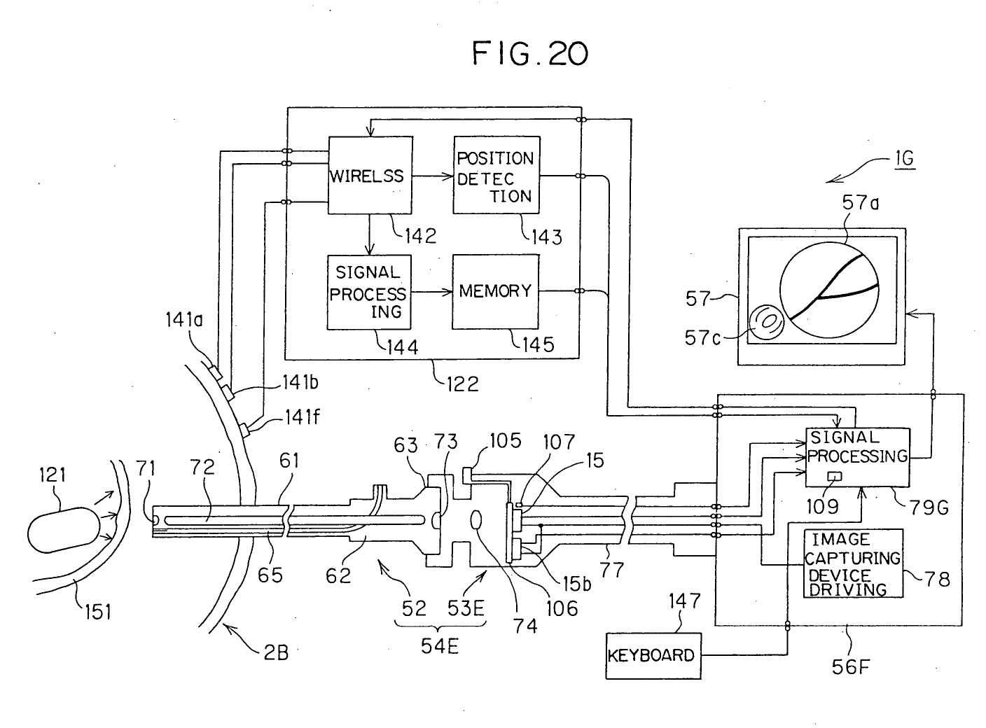

Next, an infrared observation system 1G according to a fourth embodiment of the present invention will be described with reference to FIG. 20. With the infrared observation system 1E according to the third embodiment illustrated in FIG. 18, the infrared observation system 1G according to the present embodiment does not include the light source device 55E, but instead of this, includes a capsule-type endoscope 121 to be disposed at the inside of a body cavity, and an external device 122, which is disposed at the outside of the body, for performing (two-way) wireless communication with the capsule-type endoscope 121, and also performing detection of the position of the capsule-type endoscope 121, and so forth.

Also, with the CCU 56F according to the present embodiment, the CPU 109 is connected to the external device 122 in the CCU 56E in FIG. 18, and upon the switching lever 105 being operated, the CPU 109 controls the illumination state by illumination means (radiation means) provided in the capsule-type endoscope 121 via the external device 122 instead of controlling the illumination state of the light source device 55E in FIG. 18. That is to say, the CPU 109 controls the capsule-type endoscope 121 via the external device 122 so as to assume the illumination state corresponding to the switching operation of the switching lever 105.

Accordingly, the external device 122 also has a function for wirelessly transmitting a control signal to the capsule-type endoscope 121 under control of the CPU 109.

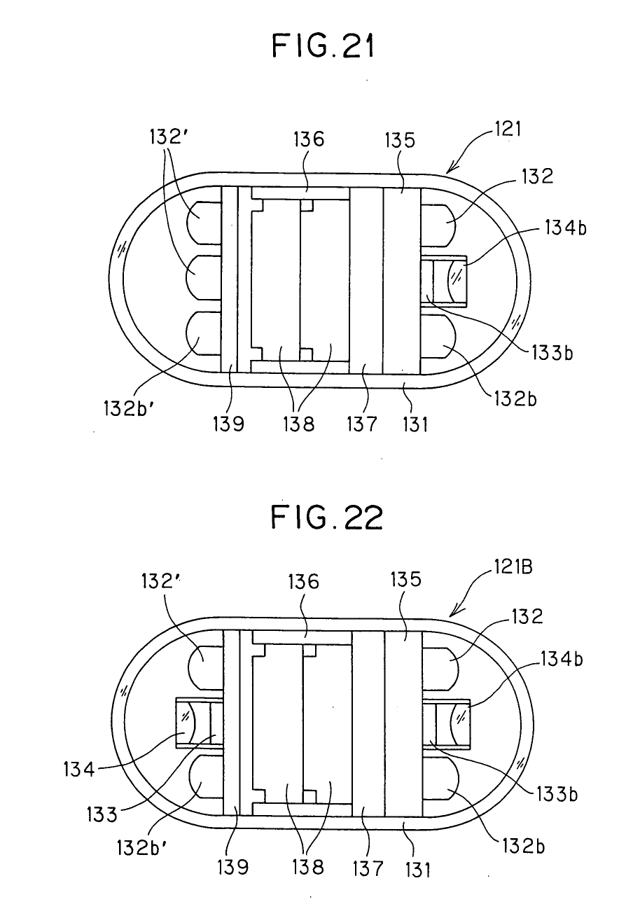

As illustrated in FIG. 21, with the capsule-type endoscope 121, a capsule-shaped airtight container 131 stores an infrared LED 132 serving as infrared light illumination means for performing illumination toward the outside of the body from the inside of the body, a white LED 132b serving as normal light illumination means which is employed as illumination for capturing an image at the capsule-type endoscope 121, and also can be employed for illumination toward the outside of the body from the inside of the body, and an image capturing device 133b for performing ordinary light image capturing, for example.

Specifically, at least both end side portions in the capsule-shaped airtight container 131 are made up of a semi-spherical-shaped transparent member. FIG. 21 transparentizes the entire airtight container 131. The lens frame to which an objective lens 134b is attached is disposed around the center of the inside of one end portion, and an image capturing device 133b is disposed at the image-formation position of the objective lens 134b. For example, multiple infrared LEDs 132 and white LEDs 132b are disposed around the image capturing device 133b.

Also, within the airtight container 131 a board to which a lens frame, the infrared LED 132, and the white LED 132b are attached is disposed, the board controls ON/OFF of the infrared LED 132 and the white LED 132b, and also makes up a control circuit 135 for performing signal processing as to the image capturing device 133b.

Also, within the airtight container 131 a wireless circuit 137 for wirelessly transmitting the signal which has been subjected to signal processing at the control circuit 135 using an antenna 136, and a battery 138 for supplying electric power to the LEDs 132 and 132b, image capturing device 133b, control circuit 135, and wireless circuit 137 are stored.

Also, a board 139 for attachment is disposed at the inside of the end portion of the opposite side as to one end portion side where the image capturing device 133b is disposed, and the board 139 also has multiple infrared LEDs 132′ and white LEDs 132b′ attached for performing illumination toward the outside of the body from the inside of the body. ON/OFF control of the LEDs 132′ and white LEDs 132b′ is also performed by the control circuit 135.

The infrared LEDs 132 and 132′ are made up of means having properties of emitting light (lighting) in a specific wavelength band alone, as described with the first embodiment. Thus, in the event of performing image capturing in an infrared band by the image capturing device 15 serving as infrared image capturing means in the scope 54E, the present embodiment also provides identifying means, as the wavelength band of light employed for the image capturing, so as to identify the difference due to moisture extinction properties between the case of blood or blood vessel and the case of the other living body tissues.

Note that thus, the infrared illumination means side is not restricted to being set to such a specific wavelength band, but an arrangement may be made wherein infrared illumination means for emitting light, for example, at a broadband in infrared light is employed in the infrared LEDs 132 and 132′, and the filter 13 for transmitting light having a specific wavelength band is attached to, for example, the image capturing surface of the image capturing device 15 at the scope 54E side.

With the capsule-type endoscope 121, in the ordinary operation mode (capsule image capturing mode), the control circuit 135 turns on the white LED 132b disposed around a position adjacent to the image capturing device 133b in a certain cycle, the lighting thereof illuminates the visual field range of the image capturing device 133b, and the image capturing device 133b performs operation of ordinary illumination and ordinary image capturing.

In this case, the image data captured by the image capturing device 133b is modulated in the wireless circuit 137, and is wirelessly transmitted to the outside. The external device 122 receives the transmitted image data by antennas 141a through 141f, detects the position of the capsule-type endoscope 121 by a position detection circuit 143, and also generates image data using a signal processing circuit 144, and sequentially stores the image data in memory 145.

Upon receiving an infrared illumination control signal from the external device 122, the control circuit 135 performs control for turning on the infrared LEDs 132 and 132′ for a certain period.

Also, upon receiving a ordinary illumination control signal from the external device 122, the control circuit 135 performs control for turning on the white LEDs 132b and 132b′ for a certain period.

When the infrared LEDs 132 and 132′ each disposed at both end sides are turned on, around the capsule-type endoscope 121 can be illuminated in a broad range by infrared light. Even when the white LEDs 132b and 132b, are turned on, around the capsule-type endoscope 121 can be illuminated in a broad range by white light (visible light) in the same way.

Also, as illustrated in FIG. 20, the external device 122 includes a wireless circuit 142, which is connected to the multiple antennas 141a through 141f to be disposed on the body surface of a patient or the jacket wore by a patient using an attachment member or the like, having a function for receiving the signal to be wirelessly transmitted from the antenna 136 of the capsule-type endoscope 121 by the antennas 141a through 141f, subjecting the signal to demodulation or the like, and transmitting a control signal for switching illumination light.

The wireless circuit 142 transmits the received signal to the position detection circuit 143, the position detection circuit 143 detects (estimates) the position of the capsule-type endoscope 121 based on the signal intensity received by the multiple antennas 141a through 141f, and transmits the position information to the CPU 109 in the CCU 56F.

Also, the wireless circuit 142 demodulates the received signal, transmits this to the signal processing circuit 144, the signal processing circuit 144 sequentially stores the image digital data captured by the image capturing device 133b in nonvolatile memory 145, such as flash memory, EEPROM, or the like for example, serving as image recording means.

The memory 145 is also connected, for example, to the CPU 109 of the CCU 56F. An arrangement is made wherein in response to the instructions made by a surgeon or the like, the CPU 109 can fetch the image data stored in the memory 145, and display the image captured by the image capturing device 133b of the capsule-type endoscope 121 on the monitor 57.

For example, the surgeon can display an image 57c captured by the capsule-type endoscope 121 on the display screen of the monitor 57 by operating a keyboard 147 connected to the CCU 56F to perform inputting instructions for displaying the image data stored in the memory 145 as to the CPU 109.

The information of the position of the capsule-type endoscope 121 detected by the position detection circuit 143 is input in the CPU 109 as to the reference position which has been set beforehand to around the tip of the scope 54E. The CPU 109 receives the information of the detected position, and calculates the distance between the reference position and the detected position. Subsequently, the CPU 109 displays the calculated distance on the monitor 57 via the signal processing circuit 79E.

Also, upon the calculated distance reaching within a predetermined value which has been set beforehand, the CPU 109 performs control so as to notify, for example, on the monitor 57 that the capsule-type endoscope 121 reaches an available state for illumination. This notification may be made with characters, or an arrangement may be made wherein an infrared light illumination mark or the like is displayed on the monitor 57, the portion thereof is colored and displayed with a specific color such as green or the like, thereby notifying the surgeon that the capsule-type endoscope 121 is available for infrared illumination.

Also, upon a switching operation being operated by the switching lever 105, the CPU 109 detects the switching operation by the output from the position sensor 107, and transmits a control signal for performing illumination corresponding to the image capturing state subjected to switching setting to the wireless circuit 142 of the external device 122.

Actions according to the present embodiment having such a configuration will be described below. For example, as illustrated in FIG. 20, the surgeon inserts the insertion portion 61 of the scope 54E into the abdomen 2B via an unshown trocar, and sets this to the position to intend to perform treatment such as an incision or the like, e.g., around the position facing the outer wall of stomach 151.

Also, the surgeon uses the display on the monitor 57 or the like, and in the event that the capsule-type endoscope 121 is available for illumination, specifically in the event that the capsule-type endoscope 121 which a-patient swallowed from the mouth reaches the inside of the stomach 151, the surgeon switches the switching lever 105 to a state for performing infrared image capturing.

Then, the CPU 109 recognizes the switching operation from the output signal from the position sensor 107, sets the signal processing circuit 79F to a signal processing state as to the infrared light image capturing device, and also transmits the control signal of a command to the wireless circuit 142 of the external device 122 for having it perform infrared light illumination.

This control signal is wirelessly transmitted from, for example, the antenna 141a by an electric wave, and the capsule-type endoscope 121 demodulates this control signal via the antenna 136 and wireless circuit 137, and transmits the demodulated control signal to the control circuit 135.

The control circuit 13 decodes the content of the demodulated control signal by collating this with the code stored in unshown memory within the control circuit 13 beforehand, or the like. Subsequently, upon decoding that this is an infrared light illumination command, the control circuit 135 turns on the infrared LEDs 132 and 132′.

Upon the infrared LEDs 132 and 132′ being turned on, an image is captured by the image capturing device 15 of the scope 54E using the light having a specific wavelength band in the infrared light which is transmitted through the wall of the stomach 151. Subsequently, the image capturing signal captured by the image capturing device 15 is subjected to signal processing at the signal processing circuit 79F, converted into a picture signal, and the image 57a captured at the image capturing device 15 is displayed on the monitor 57.

According to this display, the surgeon can recognize the course of a blood vessel where blood is flowing around the inner wall of the stomach 151. In the event of attempting to perform treatment such as incision or the like, the surgeon can perform treatment such as incision or the like smoothly by suppressing bleeding with reference to the image of the course of the blood vessel.

Also, in the event that the wall surface is thin, the surgeon may switch to a state for observing this using the ordinary light image capturing device 15b by operating the switching lever 105. In this case, the CPU 109 transmits the control signal of a command for performing ordinary light illumination to the capsule-type endoscope 121 via the external device 122.

Subsequently, the control circuit 135 of the capsule-type endoscope 121 turns on the white LEDs 132b and 132b′ serving as ordinary light illumination means. Subsequently, upon the white LEDs 132b and 132b, being turned on, an image is captured by the image capturing device 15b of the scope 54E using the light which is transmitted through the wall of the stomach 151. The surgeon can also observe the image captured with ordinary light by displaying on the monitor 57 the image captured by the image capturing device 15b.

According to the present embodiment, infrared illumination is performed from the inside of a body cavity which cannot be easily performed with an ordinary endoscope device, an observation image is obtained using transmission light which is transmitted through an observation object portion by using a wavelength band wherein extinction properties characteristically differ between the case of blood and the case of the other tissues.

In this case, the portion to be the deep portion side of the observation object portion is illuminated from the scope 54E side for performing image capturing in a state wherein the amount of illumination light is greater than that at the surface layer side, so the image information at the deep portion side can be obtained in a higher S/N state compared with the case of employing reflection light. Also, even in this case, an image, which has contrast difference between the case of blood or blood vessel and the case of other living body tissues, and which can be readily identified, can be obtained.

Also, the image captured by the capsule-type endoscope 121 is also displayed, thereby obtaining further detailed image information, and consequently, treatment such as an incision or the like, diagnosis, or the like can be readily performed.

FIG. 22 illustrates a capsule-type endoscope 121B according to a modification of the fourth embodiment. With the capsule-type endoscope 121B, the capsule-type endoscope 121 in FIG. 21 is modified wherein a lens frame having the objective lens 134 attached is disposed at the center position of the board 139, and the infrared light image capturing device 133 is disposed at the image-formation position of the objective lens 134.

That is to say, while the capsule-type endoscope 121 in FIG. 21 performs operation of ordinary illumination and ordinary image capturing in the ordinary operation mode, the capsule-type endoscope 121B performs both of operation of ordinary illumination and ordinary image capturing, and operation of infrared illumination and infrared image capturing, alternately.

The infrared LED 132′ is turned on at the time of infrared illumination and infrared image capturing, and this lighting causes the image capturing device 133 to perform image capturing. Subsequently, the captured image data is transmitted to the external device 122, and is stored in the memory 145 of the external device 122.

Also, in the event of the switching lever 105 being operated, the same operation as the case of the capsule-type endoscope 121 is performed. According to the present modification, the infrared image information by the capsule-type endoscope can be further obtained. Also, the image thereof can be displayed on the monitor 57. Accordingly, with the capsule-type endoscope 121B, much more image information can be obtained than the case of the capsule-type endoscope 121 in FIG. 21, thereby facilitating appropriate diagnosis and so forth.

Note that in the event that the orientation in the longitudinal direction of the capsule-type endoscope 121 or 121B can be detected by increasing the number of the antennas 136 within the capsule-type endoscope 121 or 121B, or the like, and the illumination means of the capsule-type endoscope 121 or 121B is turned on for the sake of illumination for the scope 54E, control may be made so as to turn on only the illumination means at the side facing the scope 54E side.

Alternatively, in the case of turning on each of the illumination means disposed at the both end sides within the capsule-type endoscope 121 or 121B, by alternately turning on one of the illumination means, or the like, control may be made so as to turn on only one of the illumination means which can effectively perform illumination based on the luminance level of the output signal of the image capturing means at the scope 54E side in that case.

### Fifth Embodiment

Next, an infrared microscope system 1D according to a fifth embodiment of the present invention will be described with reference to FIG. 23. The infrared microscope system 1D includes a light source device (illumination device) 82 for irradiating illumination light having an infrared wavelength band at a spacemen 81 serving as removed living body tissue, a microscope main unit 83 for solid observation by receiving, for example, the transmission light from the spacemen 81 at which illumination light is irradiated, and enlarging and observing this, and a signal processing device 84 for performing signal processing as to left and right image capturing devices 95a and 95b for infrared image capturing provided within the microscope main unit 83. The picture signal subjected to signal processing by the signal processing device 84 is displayed on left and right display elements 85a and 85b for image display disposed within the microscope main unit 83.

The light source device 82 includes a lamp 87 such as a halogen lamp or the like which is turned on by lamp lighting electric power supply from a lamp lighting circuit 86, the filter 13 for transmitting light having a specific wavelength band as described with the first embodiment, and a condenser lens 88 for condensing the infrared light which is transmitted through the filter 13. The light condensed at the condenser lens 88 is reflected at a mirror 89, and irradiated at the back face side of the spacemen 81.

Also, the infrared light which has been irradiated from the back face side and transmitted through the spacemen 81 is cast into an objective lens 91 having a great bore diameter provided in the microscope main unit 83, passes through relay lenses 92a and 92b disposed so as to be spaced left and right from the optical axis of the objective lens 91, and an image based on the infrared light is formed at the left and right image capturing devices 95a and 95b for infrared image capturing each disposed at an image-formation position. Note that as for the image capturing devices 95a and 95b, image capturing devices having sensitivity in an infrared band as with the above image capturing device 15 are employed.

The left and right image capturing signals subjected to photoelectric conversion by the image capturing devices 95a and 95b are each input to signal processing circuits 84a and 84b, and are each subjected to signal processing to generate left and right picture signals. These left and right picture signals are output to the display elements 85a and 85b constituted of a liquid crystal display element or the like respectively, and the left and right images captured at the image capturing devices 95a and 95b are displayed using the display elements 85a and 85b.

Left and right ocular windows facing the left and right display elements 85a and 85b respectively have ocular lenses 93a and 93b attached, and a user such as a surgeon or the like can perform solid observation of the spacemen 81 by observing the image of the spacemen 81 enlarged and displayed on the display elements 85a and 85b via the ocular lenses 93a and 93b through both eyes.

According to the present embodiment, as with the cases of the first and second embodiments, an image can be observed as a solid image which has a contrast difference depending on the difference of moisture content between the case in which the deep portion of the living body tissue of the spacemen 81 is blood or blood vessel and the case of other living body tissues, and can be readily identified.

Also, with the signal processing device 84, an image which can be further readily identified can be obtained by using at the same time the image processing described with the first embodiment.

Note that with the present embodiment, description has been made in the case of performing solid observation of the removed living body tissue of the spacemen 81 for example, but the present embodiment can be applied to the case of performing directly solid observation of a living body tissue from the outside of the body. In this case, an applicable range is expanded using reflection light, but a thin portion such as a hand, finger, or the like can be observed using transmission light.

### Sixth Embodiment

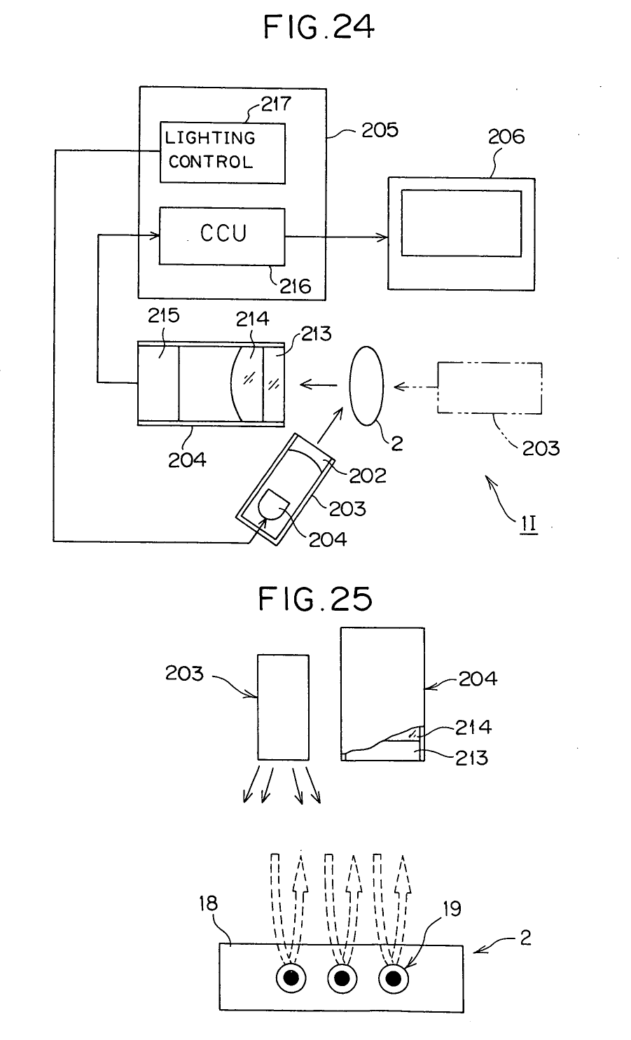

Next, a sixth embodiment of the present invention will be described with reference to FIGS. 24 through 30.

As illustrated in FIG. 24, an infrared observation system 1I according to the sixth embodiment of the present invention comprises: a light source device 203 for irradiating light including an infrared region as to a living body 2 for example serving as a subject, as illumination light; an infrared image capturing camera (hereinafter, simply abbreviated as infrared camera) 204 for performing image capturing using light reflected at the living body 2 on which the illumination light is irradiated, or light of an infrared region in the light which has been transmitted such as illustrated in a two-dot chain line; a control device 205 for performing signal processing as to the image capturing signal captured by the infrared camera 204; and a monitor 206 for displaying the picture signal to be output from the control device 205.

The light source 203 incorporates a lamp 211 such as a halogen lamp, tungsten lamp, or the like for generating illumination light from a visible region through an infrared region exceeding at least a wavelength of 1200 nm, for example.

Also, the lamp 211 preferably has a great emission intensity in a later-described specific narrowband wavelength band. As for the lamp 211, a halogen lamp having continuous emission properties up to a wavelength band exceeding 3000 nm for example can be employed.

The illumination light using lighting of the lamp 211 is irradiated at the living body 2 via the illumination lens 212. An image based on the reflection light at the time of the living body 2 being irradiated is formed on the image capturing surface of an image capturing device 215, the reflection light being transmitted through a filter 213 constituting the infrared camera 204 serving as infrared image capturing means having sensitivity in an infrared region exceeding at least a wavelength of 1200 nm, and an image-formation lens 214.

Note that in FIG. 24, the infrared camera 204 is illustrated with a configuration wherein when illumination light is irradiated at the living body 2 from the light source device 203, the reflection light from the living body 2 is received, but an arrangement may be made wherein the light source device 203 is disposed such as illustrated in a two-dot chain line for example, and the transmission light transmitted by the living body 2 is received.

The image capturing device 215 employed for the above infrared camera 204 is an image capturing device made up of a semiconductor detecting device (photovoltaic semiconductor detecting element), for example, such as Ex. InGaAs, InAs, InSb, or the like having sensitivity in an infrared wavelength band exceeding at least a 1200-nm wavelength. These image capturing devices have sensitivity in a wavelength band at least from 1200 nm to 2550 nm or so. Note that InAs and InSb have sensitivity even as to a long wavelength equal to or longer than 3000 nm which is longer than 2550 nm.

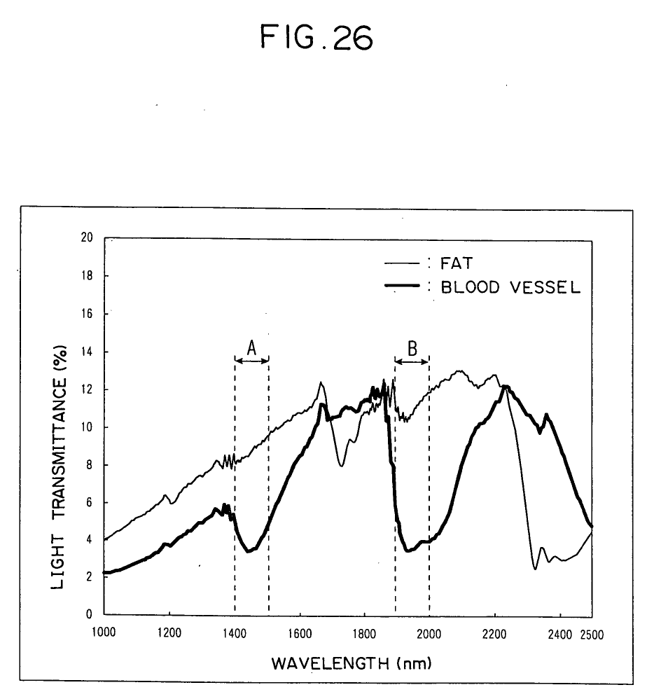

Also, as illustrated in FIG. 26, the filter 213 disposed in front of the image capturing device 215 is set to a specific narrowband wavelength so that the transmittance of a blood vessel exhibits the minimum value in a wavelength region exceeding a wavelength of 1200 nm, in other words, a wavelength wherein the absorptivity of a blood vessel exhibits a peak is transmitted in a narrowband.

Specifically, the filter 213 is set to transmission properties for transmitting one of a first narrowband wavelength band for transmitting 1450 nm±50 nm (illustrated in a symbol A in FIG. 26), and a second narrowband wavelength band for transmitting 1950 nm±50 nm (illustrated in a symbol B in FIG. 26).

That is to say, with the present embodiment, the image capturing light to be cast into the image capturing device 215, which is disposed at the image capturing means side, for capturing an image (receiving light) to obtain image information is set so as to become a specific narrowband wavelength. Like the case of a later-described seventh embodiment, an arrangement may be made wherein the wavelength band of the image capturing light to be captured by the image capturing device 215 is set so as to become a specific narrowband wavelength by restricting the wavelength band of illumination light at the illumination side.

Also, these narrowband wavelength bands are selectably set so as also to be a wavelength band wherein the transmittance as to fat tissue is a sufficiently great value, as illustrated in FIG. 26. Specifically, with the first narrowband wavelength band and the second narrowband wavelength band, the value of the transmittance of fat is double or more as compared with the value of the transmittance of a blood vessel.

Note that the absorption peak of a blood vessel at the second narrowband wavelength band side is wider than the case of the first narrowband wavelength band, so the transmission wavelength range by the filter 213 may be set wider than the case of the first narrowband wavelength band side.

Note that as for a specific narrowband wavelength by the first narrowband wavelength band and the second narrowband wavelength band, for example, a case wherein the transmission wavelength range is set to a narrowband of around 100 nm is shown, but the present embodiment is not restricted to this, any transmission wavelength range may be set as long as it is set to a narrowband in a range of several 10 nm through 100 nm or so. According to the transmission wavelength range thus set, in the event of desiring to recognize the course state of a blood vessel under a state in which the blood vessel is covered with fat as described with later-described actions, image capturing can be performed in a high S/N state in the event that a fat tissue portion has light transmit with little attenuation, and the light reflected at a blood vessel tissue under the fat tissue is subjected to image capturing.

Illumination light ranging from a visible region to an infrared region is irradiated at the living body 2, but the optical image formed at the image capturing device 215 is constituted of a specific narrowband wavelength light of the first narrowband wavelength band or the second narrowband wavelength band which is transmitted through the filter 213.

That is to say, with the present embodiment, wavelength restricting means (spectral means) for restricting the wavelength of the image capturing light is made up of the filter 213 such that the light subjected to image capturing by the image capturing device 215 is a specific narrowband wavelength light at longer wavelength side than a wavelength of 1200 nm.

The optical image formed on the image capturing surface of the image capturing device 215 is subjected to photoelectric conversion by the image capturing device 215. The signal subjected to photoelectric conversion is output from the image capturing device 215 as an image capturing signal with a driving signal being applied to the image capturing device 215 from an unshown driving circuit within a camera control unit (abbreviated as CCU) 216 built in the control device 205. This image capturing signal is input to the CCU 216, and is converted into a picture signal by an unshown picture signal generating circuit within the CCU 216.

Subsequently, this picture signal is output to the monitor 206, and the image captured by the image capturing device 215 is displayed on the display screen of the monitor 206. Also, the control device 205 lights and drives the lamp 211 within the light source device 203, and also incorporates a lighting control circuit 217 capable of controlling the amount of emission thereof.

The infrared observation system 1I according to the present embodiment thus configured has actions such as the schematic view illustrated in FIG. 25. Note that FIG. 25 illustrates the case of infrared observation using reflection light.

As illustrated in FIG. 25, illumination light ranging from a visible region to an infrared region is irradiated at the living body 2 from the light source device 203. Subsequently, the reflection light from the living body 2 is subjected to image capturing by the infrared camera 204. With the living body 2, the case of a state in which the blood vessel 19 is covered with the tissue of the fat 18 frequently occurs.

Consequently, the amount of attenuation is great in an infrared region serving as a shorter wavelength side than a wavelength of 1200 nm or so as well as the case of illumination light of a visible region, and it is difficult to capture an image with the reflection light from the blood vessel 19 at the underside of the fat 18.

Alternatively, with the present embodiment, the image capturing device 215 having sensitivity at a longer wavelength side than a wavelength of 1200 nm is employed, and also illumination light including a longer wavelength side than a wavelength of 1200 nm is irradiated as illumination light. Also, the filter 213 having properties for selectively transmitting specific narrowband wavelength light in a first narrowband wavelength band or a second narrowband wavelength band where the absorptivity as to the blood vessel 19 becomes a peak is disposed in front of the image capturing device 215.

Also, this specific narrowband wavelength light has high transmittance as to the tissue of the fat 18, so reaches the tissue of the blood vessel 19 with little attenuation. Subsequently, this light is absorbed by the tissue of the blood vessel 19, and accordingly, the reflection light from the tissue of the blood vessel 19 and the reflection light from the tissue of the fat 18 or the like around thereof greatly differ in reflection light intensity thereof (becomes reflection light). That is to say, as illustrated by the dotted line in FIG. 25, the reflection light upon which the course of the blood vessel 19 is reflected can be obtained.

Accordingly, in the event that the image capturing signal captured by the image capturing device 215 is subjected to signal processing at the CCU 216 to generate a picture signal, and displayed on the monitor 206, the image, such as illustrated in FIG. 28, can be obtained.

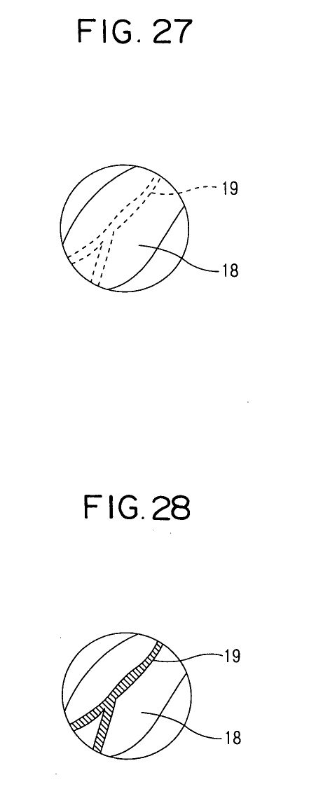

As described above, in the event of observing the blood vessel 19 covered with the tissue of the fat 18, and in the event of observing this using light in a visible region, as illustrated in FIG. 27, the attenuation is great due to the tissue of the fat 18, which is displayed as an image in which the blood vessel 19 is almost invisible such as illustrated in a dotted line.

Alternatively, in the event of observing the blood vessel 19 covered with the tissue of the fat 18 using specific narrowband wavelength light at further longer wavelength side than a wavelength of 1200 nm according to the present embodiment, this can be displayed as an image in which the blood vessel 19 has great contrast as illustrated in FIG. 28. Consequently, the course of the blood vessel 19 can be recognized from the image.

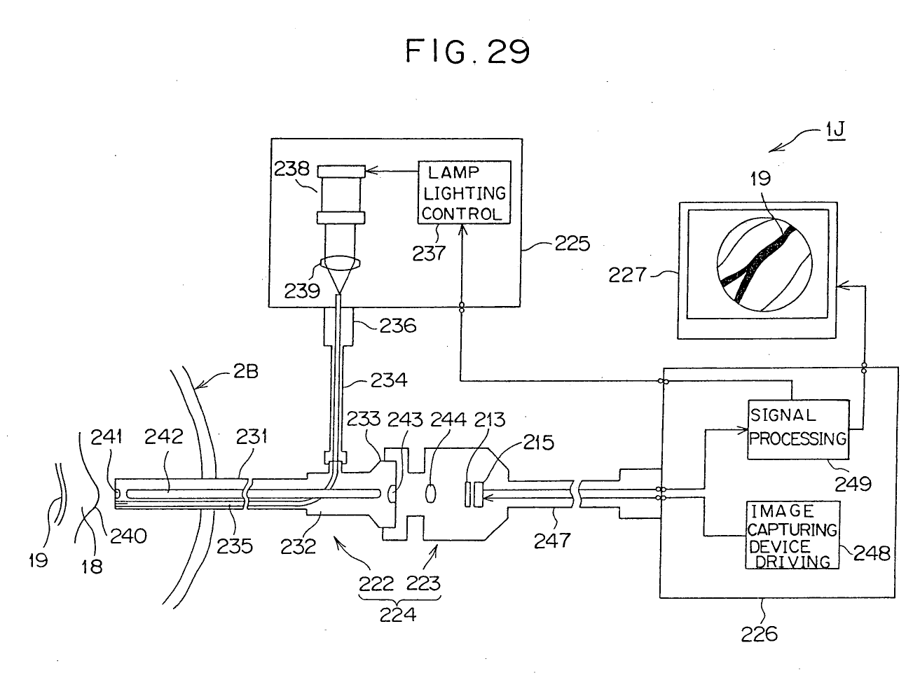

With the above description, the configuration and operation for observation using the infrared camera 204 has been described, but as illustrated in FIG. 29, an arrangement such as an infrared observation system 1J according to a first modification for observing the inside of a living body may be made.

The infrared observation system 1J according to the first modification illustrated in FIG. 29 comprises a camera mounting endoscope (hereinafter, simply abbreviated as scope) 224 mounting a camera head 223 which incorporates image capturing means within an optical endoscope 222 for example to be inserted in the abdomen 2B (of a living body 2), a light source device 225 for supplying illumination light to the optical endoscope 222, a CCU 226 for performing signal processing as to the image capturing means built in the camera head 223, and a monitor 227 for displaying the endoscope image captured by the image capturing means with the standard picture signal to be output from the CCU 226 being input.

The optical endoscope 222 includes, for example, a hard insertion portion 231, a gripper 232 provided at the back end of the insertion portion 231, and an ocular portion 233 provided at the back end of the gripper 232, and the mouthpiece of the gripper 232 is connected to a light guide cable 234.

A light guide 235 for transmitting illumination light is inserted into the inside of the insertion portion 231, and with the light guide 235, a light guide connector 236 provided at the end portion thereof is freely detachably connected to a light source device 225 via the light guide cable 234 connected to the mouthpiece of the side portion of the gripper 232.

A lamp 238 such as a halogen lamp or the like which is turned on by lamp lighting electric power to be supplied from a lamp lighting control circuit 237 is provided within the light source device 225, and the lamp 238 generates light in an infrared region exceeding at least a wavelength of 1200 nm as described above.

The light of the lamp 238 is condensed at a condenser lens 239 disposed on an illumination light path, illumination light is cast into the incident end surface of the light guide 235 of the light guide connector 236, and is transmitted to the tip surface (emitting end surface) of the insertion portion 231 by the light guide 235.

Subsequently, the illumination light is emitted from the tip surface of the light guide 235, and is emitted toward an observation object portion 240 side such as stomach or the like serving as a subject within the abdomen 2B, and illuminates the observation object portion 240.

An objective lens 241 is attached to an observation window provided adjacent to an illumination window at the tip portion of the insertion portion 231, and forms an optical image of the observation object portion such as an illuminated affected portion or the like. The optical image is transmitted to the back end surface side by a relay lens system 242 serving as an image guide.

The transmitted optical image can be enlarged and observed using an ocular lens 243 provided at the ocular portion 233. In the event that the camera head 223 is mounted on the ocular portion 233, the transmitted optical image is formed at the image capturing device 215 via an image capturing lens 244 within the camera head 223.

In this case, the filter 213, which has been set to transmission properties for transmitting one of a first narrowband wavelength band for transmitting 1450 nm±50 nm, and a second narrowband wavelength band for transmitting 1950 nm ±50 nm, is disposed within the optical path between the image capturing lens 244 and the image capturing device 215, for example.

Also, a camera cable 247 extending from the camera head 223 is connected to the CCU 226. The CCU 226 comprises an image capturing device driving circuit 248 and a signal processing circuit 249, and the image capturing device driving circuit 248 applies an image capturing device driving signal to the image capturing device 215.

Subsequently, the image capturing signal subjected to photoelectric conversion performed by the image capturing device 215 to which the image capturing device driving signal has been applied is input to the signal processing circuit 249. The signal processing circuit 249 subjects the input image capturing signal to signal processing for generating a picture signal.

Subsequently, the generated picture signal is output to the monitor 227, and the image captured by the image capturing device 215 is displayed on the display screen of the monitor 227.

Also, a dimming signal representing the mean brightness in several-frames period thereof as to the luminance level of the picture signal in the signal processing circuit 249 is input to the lamp lighting control circuit 237 within the light source device 225. Subsequently, the lamp lighting control circuit 237 controls the amount of emission of the lamp 238 by using the difference signal between the dimming signal and an unshown reference brightness signal.

Next, with the infrared observation system 1J, the actions in a case wherein surgery is performed upon the stomach to be treated within the abdomen 2B under observation using the scope 224 will be described.

With the infrared observation system 1J according to the modification, for example, in order to cut open the stomach to be treated, covered with an omentum majus or the like, which has been cancerated or the like, by inserting the insertion portion 231 of the scope 224 into the inside of the abdomen 2B via an unshown trocar as illustrated in FIG. 29, it is sometimes necessary to confirm the course of the blood vessel 19.

In this case, the omentum majus portion has the tissue of the fat 18 adhered to in the case of an adult or the like, the tissue causes the omentum majus portion to become thick, and consequently, as described above, the tissue of the fat 18 makes it difficult for the surgeon to confirm the course of the blood vessel 19 using visible light or near-infrared light.

Alternatively, in the event that the blood vessel 19 is running at the underside (inside) covered with the observation object portion 240 constituted of the tissue of the fat 18 such as an omentum majus or the like as illustrated in FIG. 29, with the present embodiment, by capturing an image using light having a specific wavelength band exceeding a wavelength of 1200 nm, the image which allows the surgeon to recognize the course of the blood vessel 19 can be obtained, such as illustrated on the monitor 227 in FIG. 29 (or FIG. 28).

Thus, even in the event that an endoscope is inserted into a body cavity to perform surgery under the endoscope, the first modification of the present embodiment allows the surgeon to recognize the course of the blood vessel 19 under the fat 18 and so forth, and perform treatment in a smooth manner and in a short period of time. Accordingly, the time for surgery can be greatly reduced, whereby both burden of a surgeon and burden of a patient can be greatly reduced.

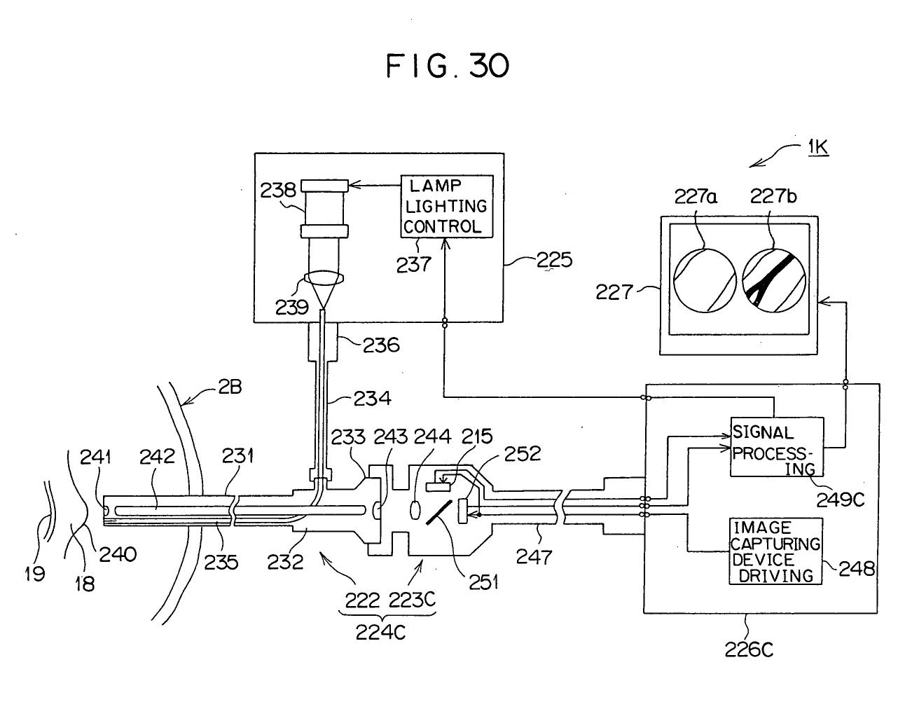

FIG. 30 illustrates an infrared observation system 1K according to a second modification. The infrared observation system 1K is configured wherein a dichroic mirror 251 serving as selective reflection means for selectively reflecting light having a specific narrowband wavelength is disposed within the camera head 223 in the infrared observation system 1J illustrated in FIG. 29, and the image capturing device 215 is disposed at the image-formation position reflected by the dichroic mirror 251.

Also, a scope 224C, which uses a camera head 223C in which an ordinary light observation image capturing device 252 constituted of a CCD or the like is disposed, is employed at an image-formation position at the transmission light side of the dichroic mirror 251. Note that the image capturing device 252 is a synchronous-type color image capturing device including an optical color separation filter such as a mosaic filter for transmitting, for example, light of R, G, and B wavelength bands in a visible region, or the like.

Also, the CCU 226C according to the present modification includes a signal processing circuit 249C having a signal processing function as to the two image capturing devices 215 and 252. The CCU 226C includes an image capturing device driving circuit 248 for driving the image capturing devices 215 and 252, and a signal processing circuit 249C for performing signal processing as to the two image capturing devices 215 and 252.

The CCU 226C according to the present modification is configured so as to mix both picture signals generated by the signal processing as to the image capturing signals of the image capturing devices 215 and 252 within the signal processing circuit 249C to be output to the monitor 227, and to display by putting side by side in the same time images 227a and 227b captured respectively by the image capturing devices 215 and 252.

Note that both of the image capturing devices 215 and 252 have the same number of pixels for example, the CCU 226C according to the present modification is configured so as to be driven commonly by the one image capturing device driving circuit 248. It is needless to say that these image capturing devices 215 and 252 may be driven individually.

According to the present modification, in addition to the image 227a for infrared observation, the color image 227b for ordinary observation in a visible region can be obtained.

Consequently, with the one scope 224C, the color image 227b for ordinary observation and the image 227a for infrared observation can be obtained, and accordingly, the surgeon can perform surgery using the one scope 224C without using multiple scopes, whereby the surgeon or the like can perform surgery in a short period of time. Accordingly, burden as to both of a surgeon and a patient can be reduced.

Note that in FIG. 30, a half prism and the filter 213 may be employed instead of employing the dichroic mirror 251.

### Seventh Embodiment

Next, a seventh embodiment of the present invention will be described.

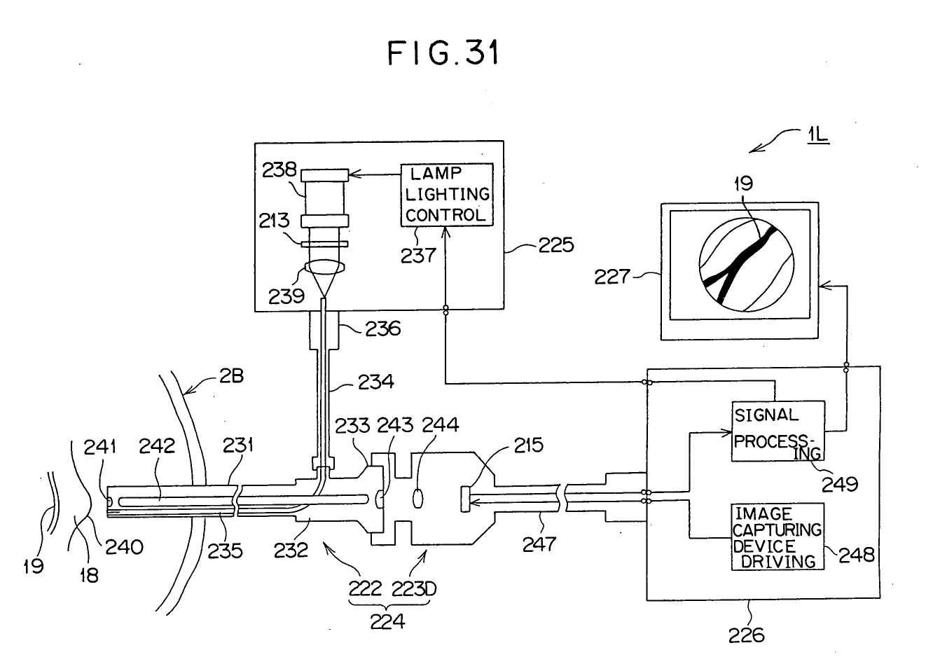

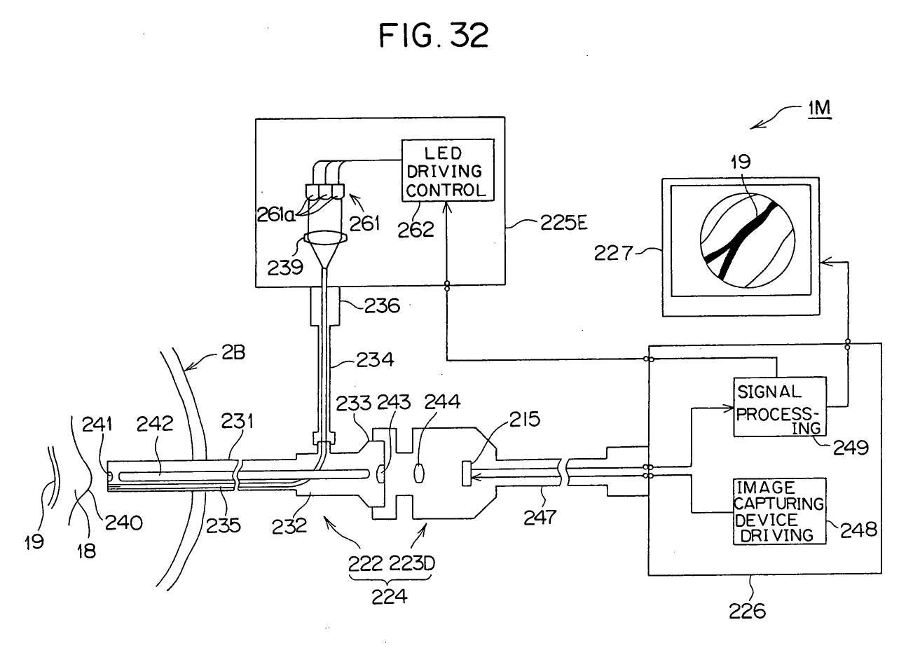

FIGS. 31 and 32 are diagrams according to the seventh embodiment of the present invention. FIG. 31 is an overall configuration diagram of an infrared observation system 1L according to the seventh embodiment, and FIG. 32 is an overall configuration diagram of an infrared observation system 1M according to a modification of the seventh embodiment.

With the sixth embodiment, an arrangement has been made wherein illumination light in a broadband, including from a visible region to a specific narrowband wavelength exceeding a wavelength of 1200 nm, is employed for irradiating the living body 2 serving as a subject, and only the light having a specific narrowband wavelength is cast into the image capturing device 215 for infrared observation using the filter 13 or the like which functions as spectral means provided at the image capturing means side.

Alternatively, with the present embodiment, the filter 213 is disposed at the light source device side, the light to be irradiated at the living body 2 serving as a subject is set so as to become a specific narrowband wavelength exceeding a wavelength of 1200 nm.

The infrared observation system 1L has a configuration obtained by modifying the filter 213 disposed within the camera head 223 in the infrared observation system 1J in FIG. 29 so as to be disposed within the light source device 225, for example.

That is to say, the infrared observation system 1L employs a camera head 223D obtained by removing the filter 213 disposed within the camera head 223 instead of the camera head 223 in the infrared observation system 1J in FIG. 29, and a light source device 225D obtained by disposing the filter 213 within the light source device 225.

Within the light source device 225D, the filter 213 is disposed on the optical path, for example, between the lamp 238 and the condenser lens 239. The other configurations are the same as those in the infrared observation system 1J in FIG. 29.

According to the present embodiment, an image from which the course of the blood vessel 19 can be recognized almost in the same way as the case in FIG. 29 according to the sixth embodiment can be obtained.

Next, a modification of the present embodiment will be described.

The infrared observation system 1M employs an infrared LED array 261 instead of the lamp 238 and the filter 213 in the light source device 225D in the infrared observation system 1L in FIG. 31, and also employs a light source device 225E having a configuration in which an LED driving control circuit 262 for driving the infrared LED array 261 to emit light is employed instead of the lamp lighting control circuit 237.

In this case, the infrared LED array 261 employs a plurality of infrared LEDs 261a having properties for emitting light with a specific narrowband wavelength exceeding a wavelength of 1200 nm as described above. The other configurations are the same as those in the case of FIG. 31.

According to the present modification, almost the same advantages as with the seventh embodiment can be obtained, and also consumption power can be reduced as compared with the case of a lamp. Also, the light source device 225E can be reduced in weight and size.

### Eighth Embodiment

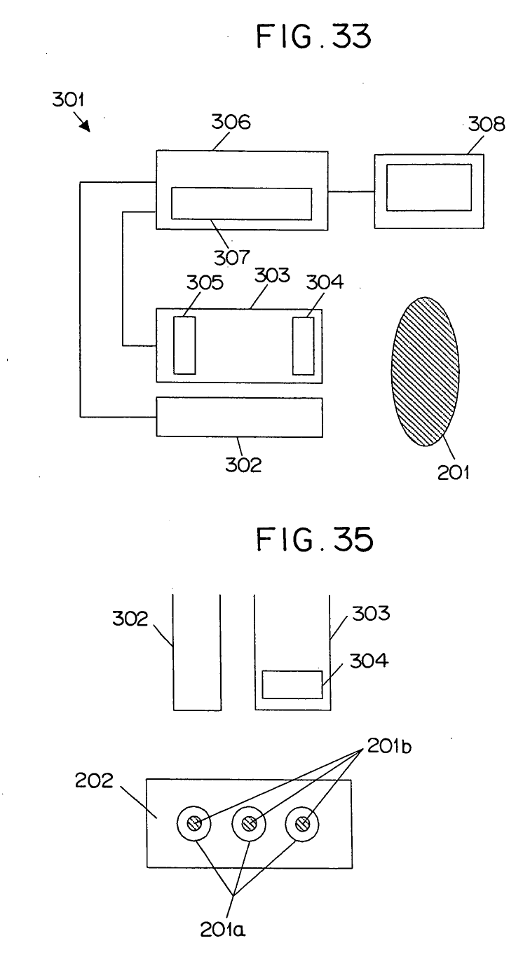

Next, an eighth embodiment of the present invention will be described with reference to FIGS. 33 through 39.

As illustrated in FIG. 33, an infrared observation system 301 comprises, as principal components, a light source device 302 serving as light source means constituted of, for example, a halogen lamp or the like serving as a light source for emitting illumination light having an infrared region band exceeding at least 1200 nm upon a subject 201 such as a living body tissue or the like, an image capturing apparatus 303 constituted of, for example, an endoscope or the like for capturing an image of the subject 201 to output the captured image of the subject 201 as an image capturing signal, and a control device 306 to be connected to the light source device 302 and the image capturing device 303.

The image capturing device 303 comprises a filter 304 serving as spectral means (or wavelength restriction means) for transmitting light having a predetermined wavelength band, and an image capturing device 305 serving as image capturing means for capturing the image of the subject 201 based on the light which is transmitted through the filter 304, and outputting the image of the subject 201 as an image capturing signal.

The filter 304 is configured so as to include the photo-absorption peak of a blood vessel as a predetermined wavelength band, and also to transmit light of a band wherein the light transmittance of fat is greater than that of the tube wall of the blood vessel. In other words, the filter 304 restricts a wavelength such that the image capturing light employed for image capturing by the image capturing device 305 for receiving the reflection light or transmission light in the illumination light emitted upon a subject becomes to have only a predetermined wavelength band. Specifically, as illustrated in FIG. 34, the filter 304 is configured so as to transmit light of a band of 1200 nm through 1600 nm, and light of a band of 1850 nm through 2200 nm, for example.

The image capturing device 305 is configured as an infrared light detection device being made up of InGaAs, InAs and InSb, or the like, for example, and having sensitivity in an infrared region exceeding a wavelength of 1200 nm.

The control device 306 includes a camera control unit (hereinafter, abbreviated as CCU) 307 for performing control as to the image capturing apparatus 303, and so forth, wherein the control device 306 generates a picture signal by performing signal processing based on the image capturing signal output from the image capturing apparatus 303 and outputs the generated picture signal to the monitor 308. Thus, on the monitor 308, the image of the subject 201 captured by the image capturing apparatus 303 based on the picture signal output from the control device 306 is displayed.

Next, description will be made regarding the actions of the infrared observation system 301.

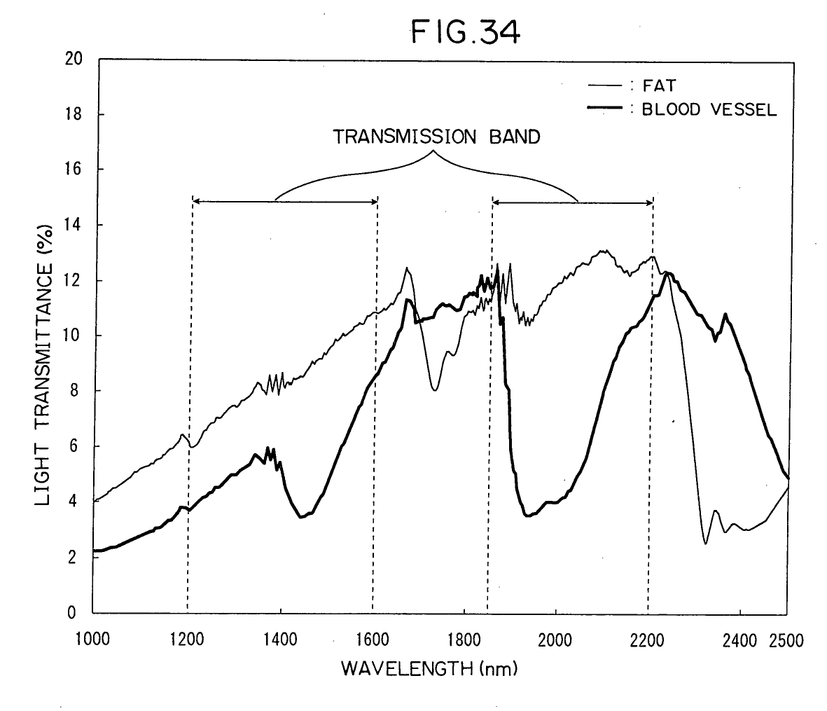

First, in order to obtain a state of a blood vessel course in the desired observation portion of a living body, the user arranges such that the light source device 302 and the image capturing apparatus 303 have the positional relation such as illustrated in FIG. 35, for example. That is to say, the light source device 302 is disposed at a position where a blood vessel 201a serving as one subject is illuminated, and also the image capturing apparatus 303 is disposed at a position where the image of the blood vessel 201a illuminated by the illumination light emitted from the light source device 302 can be captured.

Subsequently, the illumination light emitted from the light source device 302 is transmitted and reflected at the blood vessel 201a, blood 201b flowing inside the blood vessel 201a, and fat 202 covering around the blood vessel 201a. Subsequently, of the illumination light emitted from the light source device 302, the reflection light reflected at the blood vessel 201a, blood 201b, and fat 202 is cast into the filter 304.

The above reflection light cast into the filter 304 is emitted to the image capturing device 305 as light in a state in which the band components other than 1200 nm through 1600 nm and 1850 nm through 2200 nm are shielded.

The image capturing device 305 captures the image of the blood vessel 201a based on the light which is transmitted through the filter 304, and outputs the image of the blood vessel 201a as an image capturing signal.

Subsequently, the image capturing signal output from the image capturing device 305 is subjected to signal processing at the control device 306, following which is output to the monitor 308 as a picture signal.

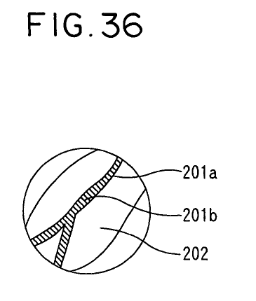

Subsequently, according to the above actions, on the monitor 308 the image such as illustrated in FIG. 36, which visualizes the blood vessel course at the blood vessel 201a covered with the fat 202, is displayed. That is to say, on the monitor 308 such an image as that the luminance of the fat 202 having high light transmittance is relatively high, and the luminance of the tube wall of the blood vessel 201a having low light transmittance is relatively low, is displayed. Thus, while viewing the image of living body tissue with the blood vessel course of a living body deep portion covered with fat and so forth which has been made clearly visible and displayed on the monitor 308, the user can perform treatment as to the relevant living body tissue in a short period of time as compared with conventional treatment.

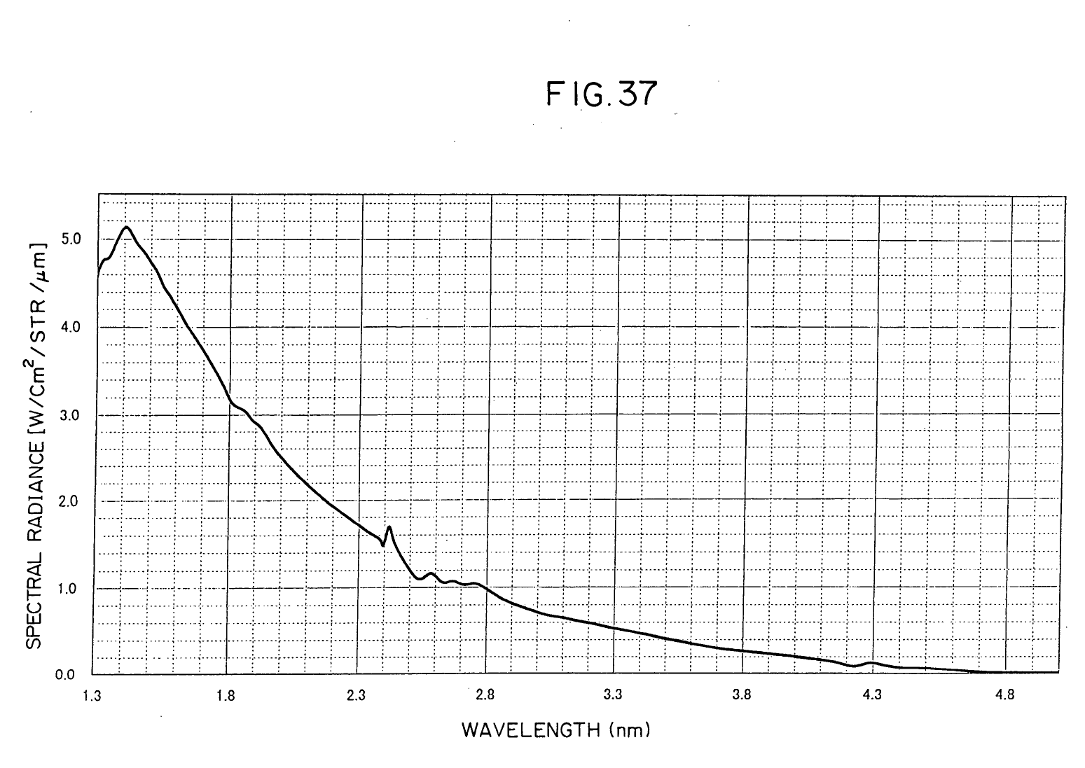

Also, as described above, even in the event of employing, as a light source in the light source device 302, a halogen lamp or the like of which irradiation luminance is attenuated at a long wavelength band equal to or longer than an infrared band with the transmission band of the filter 304 being set wide, e.g., by having properties such as illustrated in FIG. 37, the user can observe the blood vessel course of a living body deep portion while viewing an image which is displayed on the monitor 308, and has brightness suitable for observation.

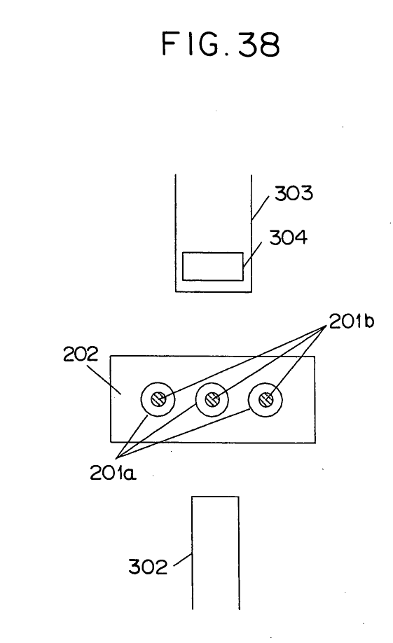

Note that the infrared observation system 301 according to the present embodiment may be a system for capturing the image of the blood vessel 201a using transmission light to obtain generally the same advantages as the above advantages. In the event of capturing the image of the blood vessel 201a using transmission light, the user should arrange such that the light source device 302 and the image capturing apparatus 303 have the positional relation such as illustrated in FIG. 38 at the desired observation portion of a living body. That is to say, the light source device 302 is disposed at a position where the blood vessel 201a is illuminated, and also the image capturing apparatus 303 is disposed at a position substantially facing the light source device 302 sandwiching the blood vessel 201a.

Also, with the infrared observation system 301 according to the present embodiment, as for a configuration for obtaining generally the same advantages as the above advantages, the filter 304 is not restricted to the one provided in the image capturing apparatus 303, e.g., the filter 304 may be provided in the light source device 302 such that illumination light having a band in which the light transmittance of fat is equal to or greater than that of the tube wall of a blood vessel is emitted at a subject.

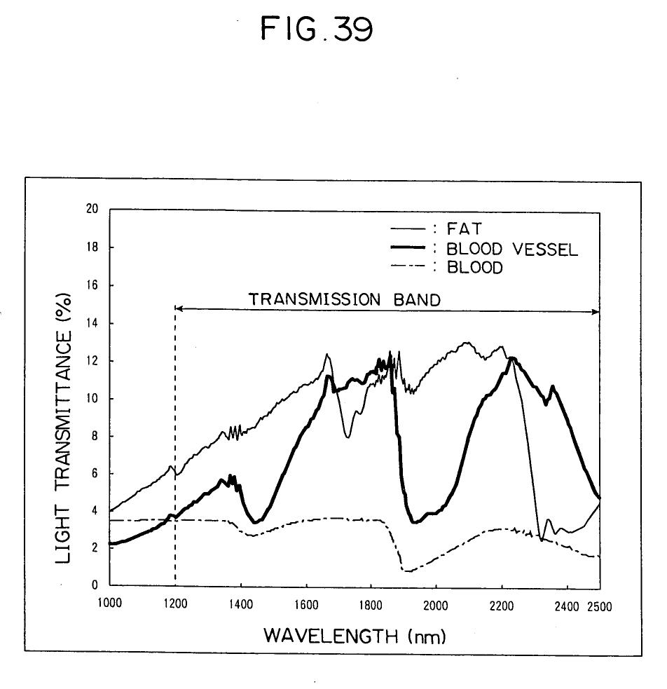

Incidentally, as illustrated in FIG. 39, the light transmittance of the blood is often less than the light transmittance of fat and the tube of a blood vessel in a band between 1200 nm and 2500 nm. Accordingly, as for a configuration for obtaining generally the same advantages as the above advantages, the infrared observation system 301 according to the present embodiment may be a system having a configuration for capturing the image based on the reflection light and transmission light in the blood 201b flowing inside the blood vessel 201a.

Specifically, the filter 304 may be a filter having a configuration for transmitting a band between 1200 nm and 2500 nm based on the above light transmittance of blood. Also, the filter 304 having a configuration for transmitting a band between 1200 nm and 2500 nm is not restricted to the one provided in the image capturing apparatus 303, e.g., may be the one provided in the light source device 302 such that illumination light having a band in which the light transmittance of fat is equal to or greater than that of blood is emitted at a subject.

### Ninth Embodiment

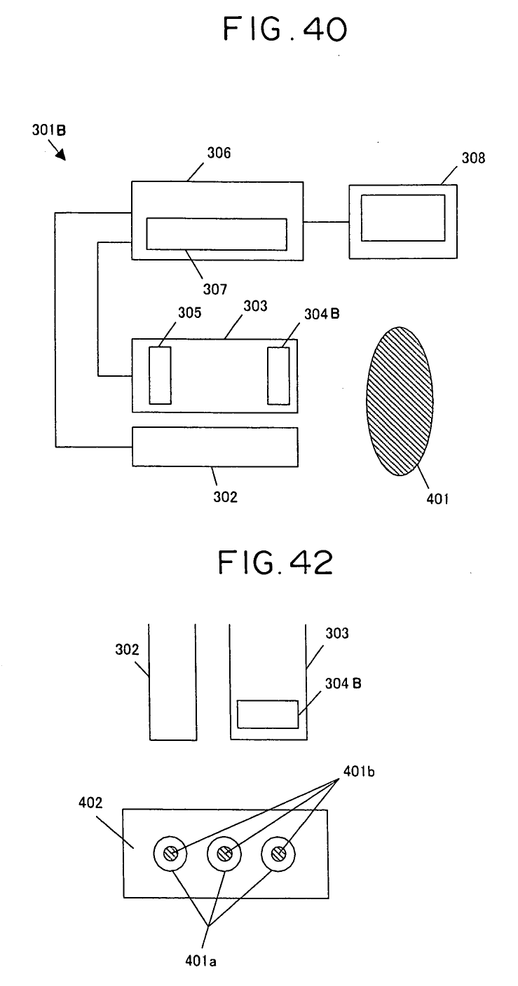

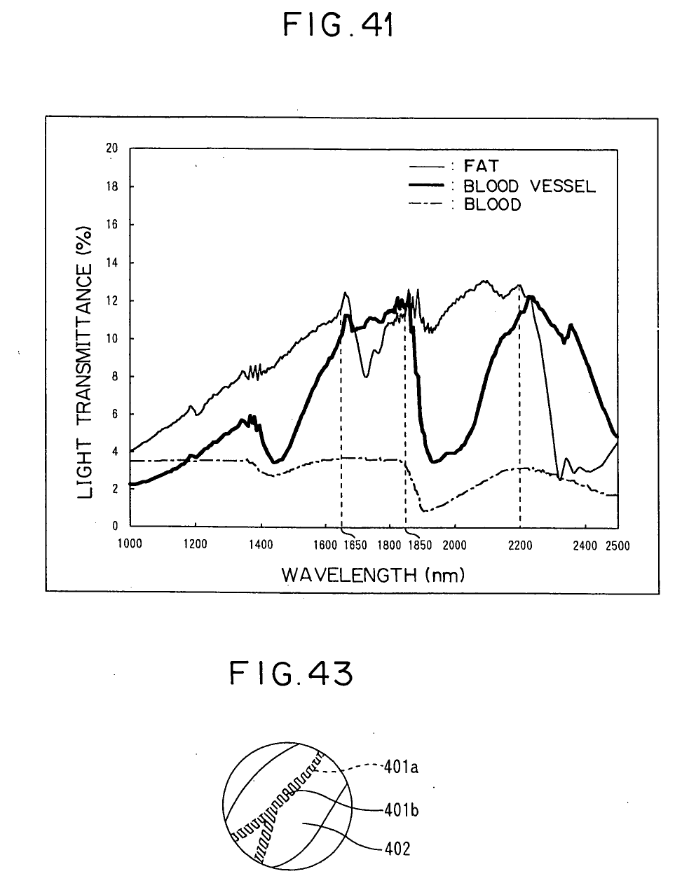

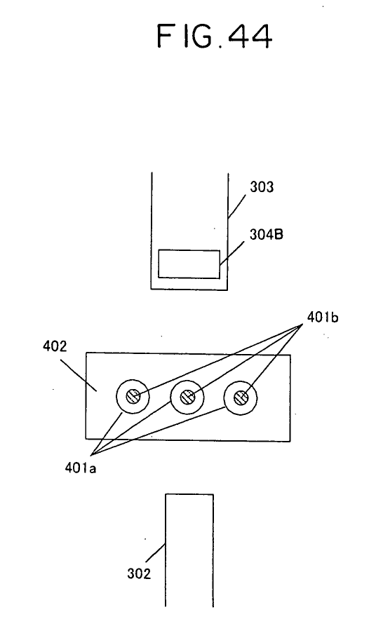

Next, a ninth embodiment of the present invention will be described with reference to FIGS. 40 through 44.

As illustrated in FIG. 40, an infrared observation system 301B comprises, as principal components, a light source device 302 serving as light source means constituted of, for example, a halogen lamp or the like serving as a light source for emitting illumination light having an infrared region band exceeding at least 1200 nm upon a subject 401 such as a living body tissue or the like, an image capturing apparatus 303 constituted of, for example, an endoscope or the like for capturing the image of the subject 401 to output the captured image of the subject 401 as an image capturing signal, and a control device 306 to be connected to the light source device 302 and the image capturing device 303.

The image capturing device 303 comprises a filter 304B serving as spectral means (or wavelength restriction means) for transmitting light having a predetermined wavelength band, and an image capturing device 305 serving as image capturing means for capturing the image of the subject 401 based on the light which is transmitted through the filter 304B, and outputting the image of the subject 401 as an image capturing signal.

The filter 304B is configured so as to transmit light having a predetermined wavelength band including a wavelength wherein the difference between the light transmittance of fat and the tube wall of a blood vessel, and the light transmittance of blood becomes the maximum, and also to shield the light having other than the predetermined wavelength band. In other words, the filter 304B restricts a wavelength such that the image capturing light employed for image capturing by the image capturing device 305 for receiving the reflection light or transmission light in the illumination light emitted upon a subject becomes to have only a predetermined wavelength band. Specifically, as illustrated in FIG. 41, the filter 304B is configured so as to transmit light having at least one band of bands of 1650±50 nm, 1850±50 nm, and 2200±50 nm for example, and also to shield the light having other than the relevant bands.

Note that with the present embodiment, for the sake of facilitating description, let us say that the filter 304B is configured so as to transmit light having a band of 1650±50 nm, and also to shield the light having other than a band of 1650±50 nm.

The image capturing device 305 is configured as an infrared light detection device being made up of InGaAs, InAs and InSb, or the like, for example, and having sensitivity in an infrared region exceeding a wavelength of 1200 nm.

The control device 306 includes a camera control unit (hereinafter, abbreviated as CCU) 307 for performing control as to the image capturing apparatus 303, and so forth, wherein the control device 306 generates a picture signal by performing signal processing based on the image capturing signal output from the image capturing apparatus 303 and outputs the generated picture signal to the monitor 308. Thus, on the monitor 308, the image of the subject 401 captured by the image capturing apparatus 303 based on the picture signal output from the control device 306 is displayed.

Next, description will be made regarding the actions of the infrared observation system 301B.

First, in order to obtain a state of a blood vessel course in the desired observation portion of a living body, the user arranges such that the light source device 302 and the image capturing apparatus 303 have the positional relation such as illustrated in FIG. 42, for example. That is to say, the light source device 302 is disposed at a position where the blood 401b serving as one subject flowing inside the blood vessel 401a is illuminated, and also the image capturing apparatus 303 is disposed at a position where the image of the blood 401b illuminated by the illumination light emitted from the light source device 302 can be captured.

Subsequently, the illumination light emitted from the light source device 302 is transmitted and reflected at the blood vessel 401a, blood 401b flowing inside the blood vessel 401a, and fat 402 covering around the blood vessel 401a. Subsequently, of the illumination light emitted from the light source device 302, the reflection light reflected at the blood vessel 401a, blood 401b, and fat 402 is cast into the filter 304B.

The above reflection light cast into the filter 304B is emitted to the image capturing device 305 as light in a state in which the band components other than 1650±50 nm are shielded.

The image capturing device 305 captures the image of the blood 401b based on the light which is transmitted through the filter 304B, and outputs the image of the blood 401b as an image capturing signal.

Subsequently, the image capturing signal output from the image capturing device 305 is subjected to signal processing at the control device 306, following which is output to the monitor 308 as a picture signal.

Subsequently, according to the above actions, on the monitor 308 the image such as illustrated in FIG. 43, which visualizes the state of the blood 401b flowing inside the blood vessel 401a covered with the fat 402, i.e., the blood vessel course is visualized. That is to say, on the monitor 308 such an image as that the luminance of the tube wall of the blood vessel 401a and the fat 402, which have high light transmittance, is relatively high, and the luminance of the blood 401b having low light transmittance is relatively low, is displayed.

Thus, while viewing the image of a living body tissue with the blood vessel course of a living body deep portion covered with fat and so forth becoming clearly visible, which is displayed on the monitor 308, the user can perform treatment as to the relevant living body tissue in a short period of time as compared with conventional treatment.

Also, the transmission band of the filter 304B in the infrared observation system 301B is set to such a band as described above, whereby the user can observe the blood vessel course state of a living body deep portion while viewing an image in which contrast between fat and a blood vessel, and blood is excellent.

Note that the infrared observation system 301B according to the-present embodiment may be a system having a configuration for capturing the image of the blood 401b using transmission light to obtain generally the same advantages as the above advantages. In the event of capturing the image of the blood 401b using transmission light, the user should arrange such that the light source device 302 and the image capturing apparatus 303 have the positional relation such as illustrated in FIG. 21 at the desired observation portion of a living body. That is to say, the light source device 302 is disposed at a position where the blood 401b is illuminated, and also the image capturing apparatus 303 is disposed at a position substantially facing the light source device 302 sandwiching the blood vessel 401a.

Also, with the infrared observation system 301B according to the present embodiment, as for a configuration for obtaining generally the same advantages as the above advantages, the filter 304B is not restricted to the one provided in the image capturing apparatus 303, e.g., the filter 304B may be provided in the light source device 302 such that illumination light having a band in which the difference between the light transmittance of fat and the tube wall of a blood vessel, and the light transmittance of blood becomes the maximum is emitted upon a subject. Such a configuration can be applied to any configuration in the case of capturing the image of the blood 401b using reflection light, and in the case of capturing the image of the blood 401b using transmission light.

### Tenth Embodiment

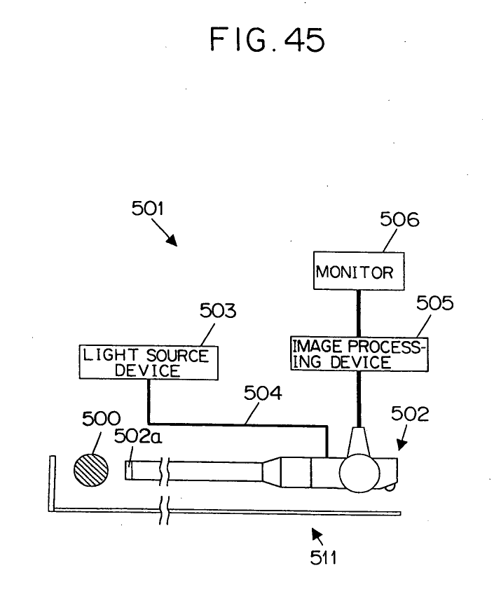

Next, a tenth embodiment of the present invention will be described with reference to FIGS. 45 through 57.

As illustrated in FIG. 45, an endoscope system 501 serving as an infrared observation system comprises, as principal components, an endoscope 502, a light source device 503 serving as a light source unit for emitting illumination light for illuminating a living body tissue 500 serving as a subject, a light guide 504 for guiding the illumination light emitted from the light source device 503 to the tip portion of the endoscope 502, an image processing device 505, a monitor 506, and a retractor 511 serving as treatment equipment.

The endoscope 502 has a configuration in which at least a part thereof is inserted into a body cavity, and also has an image capturing unit 502a at the tip portion thereof, which is constituted of an objective lens, an image capturing device, and so forth for capturing the image of the living body tissue 500, and outputting the captured image of the living body tissue 500 as an image capturing signal.

The image processing device 505 serving as an image processing unit subjects the image capturing signal output from the endoscope 502 to signal processing, and outputs this as a picture signal.

The monitor 506 serving as a display unit displays the image of the living body tissue 500 based on the picture signal to be output from the image processing device 505.

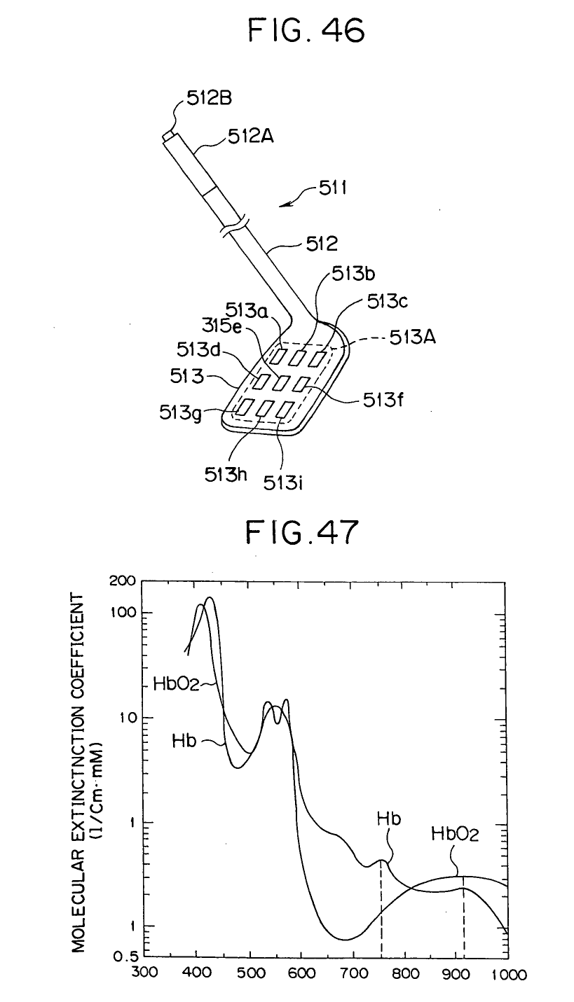

Also, as illustrated in FIG. 46, the retractor 511 comprises a slender stock portion 512, and a surface portion 513 provided at the tip portion of the stock portion 512 with an angle in the shaft direction of the stock portion 512.

The stock portion 512 comprises a gripper 512A which is gripped by a surgeon or the like in the case of operating the retractor 511, and a switch 512B provided in the gripper 512A at the back end side thereof.

The gripper 512A has an unshown power source unit constituted of an electric cell or battery or the like, and the driving current to be supplied from the power source unit turns on the LEDs provided in a later-described illumination unit (irradiation unit) 513A.

The switch 512B can switch the ON state and the OFF state of the LEDs provided in the later-described illumination unit 513A by being operated by a surgeon or the like.

The surface portion 513 comprises the illumination unit 513A in which a single or multiple surface-mounting-type LEDs for irradiating infrared light upon the subject 500 are provided. Note that with the present embodiment, as illustrated in FIG. 46, the illumination unit 513A comprises, for example, nine LEDs of LEDs 513a, 513b, 513c, 513d, 513e, 513f, 513g, 513h, and 513i serving as emission elements, but is not restricted to such a configuration.

Also, with the present embodiment, let us say that the nine LEDs included in the illumination unit 513A are configured so as to emit light having a wavelength band in the vicinity of 910 nm which is the maximum absorption wavelength in the photo-absorption properties of oxygenated hemoglobin as illustrated in FIG. 47 to observe the blood vessel course of an artery, but the LEDs are not restricted to such a configuration.

Specifically, for example, the nine LEDs included in the illumination unit 513A may be configured so as to emit light having a wavelength band in the vicinity of 760 nm which is the maximum absorption wavelength in the photo-absorption properties of hemoglobin as illustrated in FIG. 47 to observe the blood vessel course of a vein.

Next, description will be made regarding the actions according to the present embodiment.

First, a surgeon or the like connects the respective units of the endoscope system 501 in a state such as illustrated in FIG. 45, following which makes the endoscope system 501 into a starting state by turning on the power source of the respective units.

The endoscope 502 captures the image of the living body tissue 500 illuminated by illumination light emitted from the light source device 503 in a starting state, and outputs the captured image of the living body tissue 500 to the image processing device as an image capturing signal.

The image capturing signal output from the endoscope 502 is input to the image processing device 505, following which is output to the monitor 506 as a picture signal. Thus, the image of the living body tissue 500 is displayed on the monitor 506.

The surgeon or the like inserts the endoscope 502 into a body cavity up to the portion where the desired subject serving as an observation object in the blood vessel course of an artery exists while viewing the image displayed on the monitor 506. Subsequently, upon the tip portion of the endoscope 502 reaching the portion where the above-desired subject exists, the surgeon or the like inserts the retractor 511 into the body cavity via an unshown trocar or the like.

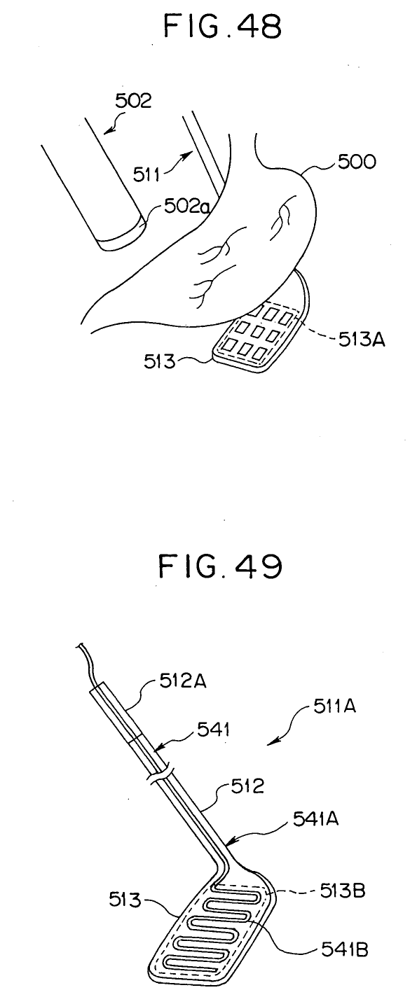

Subsequently, upon both of the tip portion of the endoscope 502 and the retractor 511 reaching the portion where the desired subject exists, the surgeon or the like arranges each of the tip portion of the endoscope 502 and the retractor 511 with respect to the living body tissue 500 serving as the above desired subject, e.g., stomach, or an omentum majus and an omentum minus and so forth fixing the stomach, so as to satisfy the positional relation such as illustrated in FIG. 48.

More specifically, the surgeon or the like moves the endoscope 502 and the retractor 511 such that the tip portion of the endoscope 502 and the illumination unit 513A of the retractor 511 are disposed at a position substantially facing each other sandwiching the living body tissue 500.

In a state in which the tip portion of the endoscope 502 and the illumination unit 513A of the retractor 511 are disposed substantially facing each other sandwiching the living body tissue 500, i.e., in a state such as illustrated in FIG. 48, the surgeon or the like stops emission of the illumination light from the light source device 503, and also brings the respective LEDs included in the illumination unit 513A into an ON state by turning on the switch 512B of the retractor 511.

In a state such as illustrated in FIG. 48, in the event that the respective LEDs included in the illumination unit 513A goes to an ON state, the image capturing unit 502a provided in the tip portion of the endoscope 502 captures the image based on transmission light which is transmitted through the living body tissue 500, and which is of the infrared light emitted from the respective LEDs included in the illumination unit 513A.

Subsequently, the image of the living body tissue 500 captured by the endoscope 502 using the transmission light of the infrared light emitted from the respective LEDs included in the illumination unit 513A is output to the image processing device 505 as an image capturing signal.

The image capturing signal output from the endoscope 502 is input to the image processing device 505, following which is output to the monitor 506 as a picture signal. Thus, the image of the living body tissue 500 in which the blood vessel course state in a deep portion (of an artery or vein) becomes more clear as compared with the image using the reflection light of infrared light is displayed on the monitor 506.

Subsequently, while viewing the image of the living body tissue 500 with the blood vessel course state in a living body deep portion which is made clear, such as described above, and which is displayed on the monitor 506, the surgeon or the like can perform treatment as to the living body tissue 500 in a short period of time as compared with conventional treatment.

Note that with the present embodiment, the retractor employed for observation using the endoscope system 501 is not restricted to the retractor having a configuration such as the retractor 511 illustrated in FIG. 46 for emitting infrared light by the LEDs included in itself being turned on, e.g., it may be a retractor having a configuration such as the retractor 511A illustrated in FIG. 49 for emitting infrared light supplied from the outside.

The retractor 511A serving as treatment equipment, which is made up of a transparent resin such as polycarbonate, comprises the stock portion 512 provided such that a fiber 541 is inserted into the inside, and a surface portion 513 having an illumination unit 513B in which one end side of the fiber 541 extending from the stock portion 512 is disposed in a waveform shape at the tip portion of the stock portion 512 with an angle in the shaft direction of the stock portion 512.

Also, the other end side of the fiber 541 extended from the stock portion 512 has a configuration which can be connected to the light source device 503 (not shown in FIG. 49). With the fiber 541 having a configuration such as described above, the illumination light emitted from the light source device 503 is supplied to the illumination unit 513B of the surface portion 513 via the stock portion 512.

Now, let us say that the above illumination light to be emitted from the light source device 503 is either the infrared light having a wavelength band in the vicinity of 910 nm for observing the blood vessel course of an artery or the infrared light having a wavelength band in the vicinity of 760 nm for observing the blood vessel course state of a vein.

Also, let us say that the light source device 503 has an unshown band restriction filter, and thus, of the above two types of infrared light, any one of the infrared light can be selectively emitted as the above illumination light.

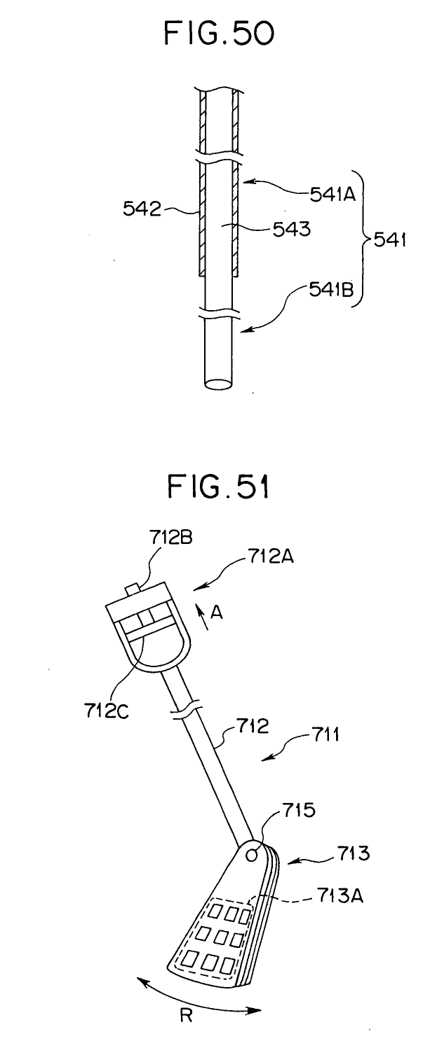

As illustrated in FIG. 50, the fiber 541 serving as an light guiding portion comprises a shielding portion 541A in a state in which a clad 543 is covered with a shielding member 542, and an emission unit 541B in a state in which the clad 543 is not covered with the shielding member 542.

As illustrated in FIG. 49, with the shielding portion 541A, one end is provided so as to be inserted into the inside of the stock portion 512, and also the other end has a configuration which can be connected to the light source device 503 (not shown in FIG. 49). Also, as illustrated in FIG. 49, the emission unit 541B constituting the illumination unit 513B is disposed in the surface portion 513 in a waveform shape with the clad 543 being exposed.

According to the above configuration, in the event that the retractor 511A is employed for observation using the endoscope system 501, the infrared light emitted from the light source device 503 is transmitted in a state shielded by the shielding portion 541A, following which is emitted to the living body tissue 500 at the emission unit 541B constituting the illumination unit 513B.

Subsequently, according to substantially the same actions as the retractor 511, as described above, the image of the living body tissue 500 in which the blood vessel course state in a deep portion (of an artery or vein) became more clear as compared with the image using the reflection light of infrared light is displayed on the monitor 506. As a result, substantially the same actions and advantage as the above case of employing the retractor 511 can be obtained.

Also, with the present embodiment, the retractor employed for observation using the endoscope system 501 is not restricted to the retractor having a configuration such as the retractor 511 illustrated in FIG. 46 or the retractor 511A illustrated in FIG. 49, e.g., it may be the retractor having a configuration such as a retractor 711 illustrated in FIG. 51.

As illustrated in FIG. 51, the retractor 711 serving as treatment equipment comprises a stock portion 712, a surface portion 713 constituted of multiple surface members attached to the tip side of the stock portion 712, and a shaft member 715 for connecting the stock portion 712 and each of the multiple surface portions.

The stock portion 712 has a gripper 712A, which is gripped by the surgeon or the like in the case of operating the retractor 711, at the back end side, and the gripper 712A comprises a switch 712B and a handle portion 712C. Also, the gripper 712A has an unshown power source unit constituted of an electric cell or battery or the like, and the driving current to be supplied from the power source unit turns on the LEDs provided in later-described illumination units 713A and 713B.

The switch 712B can be switched between the ON state and the OFF state of the LEDs provided in the later-described illumination units 713A and 713B by being operated by the surgeon or the like.

The handle portion 712C serving as a treatment equipment operating unit has an unshown retractable spring portion in the inside, and holds the position of the handle portion 712C itself so as to assume the position such as illustrated in FIG. 51 in a state in which the treatment equipment is not operated by the surgeon or the like.

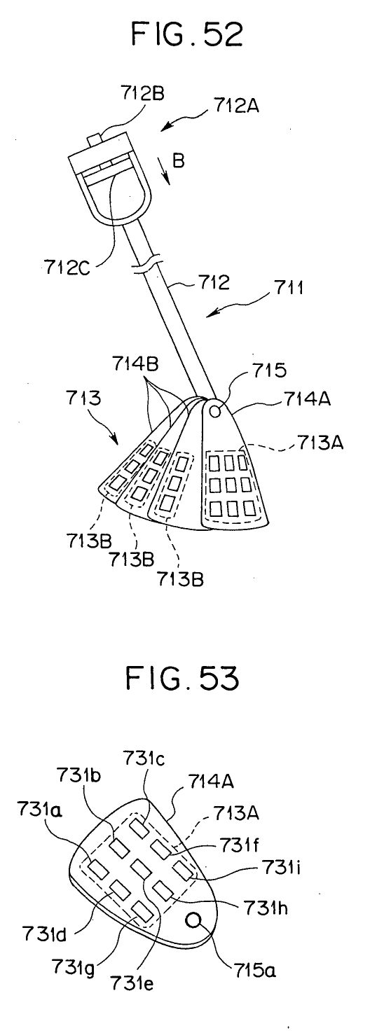

Also, the handle portion 712C has a configuration wherein upon a traction operation by the surgeon or the like in the direction illustrated in the arrow A in FIG. 51, i.e., toward the back end side of the stock portion 712, the unshown spring portion is contracted, and in addition to this action, the shape of the surface portion 713 can be changed into the fan-shaped form illustrated in FIG. 52 by later-described surface members 714A and 714B each moving rotationally in a predetermined direction with the shaft member 715 as a rotational movement shaft, i.e., in the R direction illustrated in FIG. 51.

Note that the handle portion 712C has a configuration wherein in the event of the traction operation by the surgeon or the like having been released, the unshown spring portion extends, and thus, the handle portion 712C moves in the direction illustrated in the arrow B in FIG. 52, i.e., toward the tip side of the stock portion 712, whereby the position of the handle portion 712C itself and the shape of the surface portion 713 can be returned to a state such as illustrated in FIG. 51.

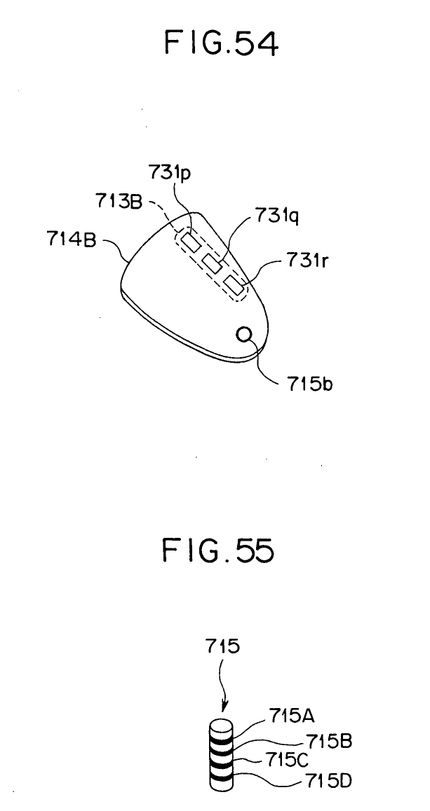

The surface portion 713 comprises one surface member 714A having a configuration such as illustrated in FIG. 52, and one or multiple surface members 714B having a configuration such as illustrated in FIG. 54. Note that with the present embodiment, the surface portion 713 is configured so as to have one sheet of the surface member 714A, and also have three sheets of the surface member 714B, but is not restricted to such a configuration.

The surface member 714A comprises the illumination unit 713A in which a single or multiple surface-mounting-type LEDs for irradiating infrared light upon a subject are provided, and a hole portion 715a having substantially the same inside diameter as the outside diameter of the shaft member 715 with an electroconductive member such as metal or the like provided on the inner circumferential surface, for example.

Note that with the present embodiment, as illustrated in FIG. 52, the illumination unit 713A is configured so as to have nine LEDs 731a, 731b, 731c, 731d, 731e, 731f, 731g, 731h, and 731i serving as emission elements, but is not restricted to such a configuration.

The surface member 714B comprises the illumination unit 713B in which a single or multiple surface-mounting-type LEDS for irradiating infrared light upon a subject are provided, and a hole portion 715b having substantially the same inside diameter as the outside diameter of the shaft member 715 with an electroconductive member such as metal or the like provided on the inner circumferential surface, for example.

Note that with the present embodiment, as illustrated in FIG. 54, the illumination unit 713B is configured so as to have three LEDs 731p, 731q, and 731r serving as emission elements, but is not restricted to such a configuration.

Note that with the present embodiment, the nine LEDs included in the illumination unit 713A and the three LEDs included in the illumination unit 713B are configured so as to emit light having a wavelength band in the vicinity of 910 nm which is the maximum absorption wavelength in the photo-absorption properties of oxygenated hemoglobin illustrated in FIG. 47 for observing the blood vessel course state of an artery, but are not restricted to such a configuration.

Specifically, for example, the nine LEDs included in the illumination unit 713A and the three LEDs included in the illumination unit 713B may be configured so as to emit light having a wavelength band in the vicinity of 760 nm which is the maximum absorption wavelength in the photo-absorption properties of hemoglobin illustrated in FIG. 47 for observing the blood vessel course state of a vein.

The shaft member 715 is configured so as have substantially the same outside diameter as the inside diameter of the hole portions 715a and 715b, and also as illustrated in FIG. 55, the shaft member 715 has multiple electrodes each connected to the above power supply unit on the outer circumferential surface in order to supply the driving current supplied from an unshown power supply unit provided in the gripper 712A to each of the surface member 714A and the surface member 714B via each of the hole portion 715a and the hole portion 715b.

Note that with the present embodiment, as illustrated in FIG. 55, the shaft member 715 is configured so as to have four electrodes 715A, 715B, 715C, and 715D, but is not restricted to such a configuration.

With a configuration such as described above, the surface member 714A is attached to the shaft member 715 such that the hole portion 715a is disposed at the position of the electrode 715A. Also, with a configuration such as described above, the three sheets of the surface member 714B are each attached to the shaft member 715 such that the hole portion 715b is disposed at the position of the electrodes 715B, 715C, and 715D.

That is to say, the surface member 713 provided at the tip side of the stock portion 712 is configured so as to have the surface member 714A and the three sheets of the surface member 714B, which are attached to the shaft member 715 in a state such as described above.

With the above configurations, in the event that the retractor 711 is employed in a state such as illustrated in FIG. 51 at the time of observation using the endoscope system 501, substantially the same actions and advantage can be obtained as the case of employing the above retractor 511 or retractor 511A.

Further, in the event that the retractor 711 is employed in a state in which the surface portion 713 is a fan-shaped form, such as illustrated in FIG. 52 at the time of observation using the endoscope system 501, in addition to the illumination unit 713A, infrared light is emitted as to the living body tissue 500 from the illumination unit 713B, so the blood vessel course state can be obtained in a further wider range of the living body tissue 500 as compared with the retractor 511 and the retractor 511A.

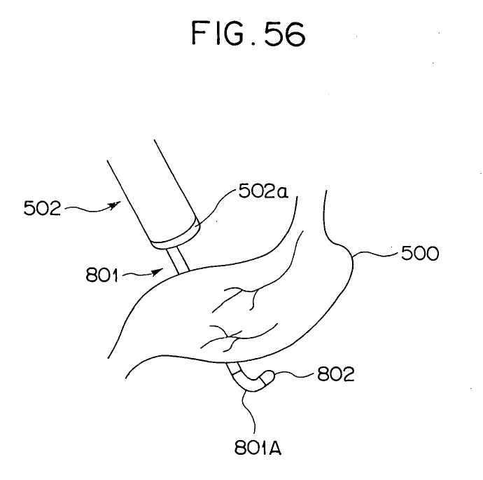

Further, in the event of employing an endoscope 502 with an unshown treatment equipment channel in the inside for inserting treatment equipment or the like at the time of observation using the endoscope system 501, the retractor 511 having a configuration such as described above can be substituted with a fiber cable 801 such as illustrated in FIG. 56, for example.

The fiber cable 801 serving as treatment equipment has a curved portion 801A which can be curved in the desired direction, and an LED 802 provided in the curved portion 801A for emitting infrared light, wherein the fiber cable 801 has a dimension and a shape which can be inserted into the unshown treatment equipment channel serving as a duct provided at the inside of the endoscope 502.

The curved portion 801A has a configuration such as described above, so can change the emission direction of infrared light from the LED 802 serving as an illumination unit.

In the event of employing the fiber cable 801 at the time of observation using the endoscope system 501, the surgeon or the like moves the endoscope 502 so as to have a state in which the tip portion of the endoscope 502 and the LED 802 are disposed substantially facing each other sandwiching the living body tissue 500, i.e., a state such as illustrated in FIG. 56, and also incurvates the curved portion 801A. Subsequently, the surgeon or the like emits infrared light from the LED 802 in a state such as illustrated in FIG. 56.

According to the above configurations, in the event of employing the fiber cable 801 at the time of observation using the endoscope system 501, substantially the above same advantages as the case of employing the retractor 511 can be obtained without employing a retractor.

Having described the preferred embodiments of the invention referring to the accompanying drawings, it should be understood that the present invention is not limited to those precise embodiments and various changes and modifications thereof could be made by one skill in the art without departing from the spirit and scope of the invention as defined in the appended claims.

What is claimed is:

1. An infrared observation system comprising:
a light source unit for generating illumination light for irradiating light including infrared light of a long wavelength exceeding at least a 1000-nm wavelength upon a living body tissue inside or outside the body in a broadband or a narrowband;
an infrared image capturing unit for capturing an image using infrared light of a wavelength band exceeding 1000 nm in the light reflected from or transmitted through the living body tissue; and
an identifying unit for facilitating identification between a case in which a living body tissue is blood or a blood vessel and a case in which a living body tissue is other living body tissue using the difference of moisture extinction properties in the wavelength band exceeding a wavelength of 1000 nm.

2. The infrared observation system according to claim 1, wherein the identifying unit is configured by setting the wavelength band of light to be received at the infrared image capturing unit in the light reflected and transmitted at the living body tissue to a specific wavelength band exhibiting a great moisture absorptivity value as the moisture extinction properties.

3. The infrared observation system according to claim 1, further comprising an image processing unit for subjecting the image capturing signal captured by the infrared image capturing unit to image processing for facilitating identification using the difference of the moisture extinction properties.

4. The infrared observation system according to claim 1, wherein the light source unit further includes a visible image capturing unit for generating illumination light having a visible light wavelength band, and performing image capturing with the visible light wavelength band based on the light reflected or transmitted at the living body tissue at which the illumination light having the visible light wavelength band is irradiated.

5. The infrared observation system according to claim 2, wherein the identifying unit includes a filter for restricting a wavelength to be transmitted such that the wavelength band of light to be received at the infrared image capturing unit in the light reflected and transmitted at the living body tissue becomes a specific wavelength band exhibiting a great moisture absorptivity value as the moisture extinction properties.

6. The infrared observation system according to claim 5, wherein the filter is disposed in front of the image capturing surface of an infrared image capturing device constituting the infrared image capturing unit, and restricts the wavelength band of light to be received by the infrared image capturing device to the specific wavelength band.

7. The infrared observation system according to claim 5, wherein the filter restricts the wavelength band of light such that the illumination light to be irradiated at a living body tissue becomes to have only the specific wavelength band.

8. The infrared observation system according to claim 1, wherein the light source unit includes an irradiation unit, which is disposed in a capsule-type endoscope, for irradiating light including infrared light having a long wavelength exceeding at least a wavelength of 1000 nm to the outside of the body from the inside of the body.

9. The infrared observation system according to claim 8, further comprising a position detecting unit for detecting the position of the capsule-type endoscope.

10. The infrared observation system according to claim 4, further comprising a switching unit for switching between image capturing using the infrared image capturing unit and image capturing using the visible image capturing unit, and controls illumination light generated at the light source unit upon a switching being made by the switching unit.

11. The infrared observation system according to claim 2, wherein the specific wavelength band includes a part of a wavelength band of 1400 nm through 1500 nm or a wavelength band equal to or greater than 1900 nm.

12. The infrared observation system according to claim 1, wherein the infrared image capturing unit is provided in an endoscope for capturing an image of the inside of the body.

13. The infrared observation system according to claim 1, wherein the infrared image capturing unit is provided in a microscope for observing a living body tissue of the outside of the body.

14. The infrared observation system according to claim 3, wherein the image processing unit includes an enhancement processing unit for performing enhancement processing in accordance with the luminance level of the image capturing signal captured by the infrared image capturing unit.

15. The infrared observation system according to claim 4, wherein the infrared image capturing unit and the visible image capturing unit can perform an image capturing action simultaneously.

16. The infrared observation system according to claim 15, wherein the infrared image capturing unit captures an image using infrared light reflected at a selective reflection unit for selectively reflecting light having the wavelength band, and the visible image capturing unit captures an image using visible light which is transmitted through the selective reflection unit.

17. The infrared observation system according to claim 4, wherein of the infrared image capturing unit and the visible image capturing unit, one switched by a switching operating unit is set to a state in which image capturing can be performed.

18. The infrared observation system according to claim 14, wherein the enhancement processing unit includes an enhancement-level determining unit for determining an enhancement level for performing enhancement.

19. The infrared observation system according to claim 14, wherein the enhancement processing unit includes a weighting coefficient value setting unit for setting a weighting coefficient value as to the amount of enhancement at the time of performing enhancement.

20. An infrared observation system comprising:
a light source unit for generating light of an infrared region having a long wavelength exceeding at least a wavelength of 1200 nm;
an infrared image capturing unit having sensitivity in an infrared region exceeding the wavelength of 1200 nm; and
a wavelength restriction unit for restricting a wavelength such that image capturing light employed for image capturing by the infrared image capturing unit for receiving reflection light or transmission light in the light irradiated at a subject exceeds the wavelength of 1200 nm, and becomes to have only a predetermined wavelength band including at least the photo-absorption peak of a blood vessel.

21. The infrared observation system according to claim 20, wherein the wavelength restriction unit, as to light emitted at the light source unit or light to be cast into the infrared image capturing unit, comprises a filter unit for transmitting only the predetermined wavelength band, or a selective reflection unit for selectively reflecting the light.

22. The infrared observation system according to claim 20, wherein the wavelength restriction unit comprises an emission element, which is provided in the light source unit, for emitting light having the predetermined wavelength band.

23. An infrared observation system comprising:
a light source unit for generating light of an infrared region having a long wavelength exceeding at least a wavelength of 1200 nm;
an infrared image capturing unit having sensitivity in an infrared region exceeding the wavelength of 1200 nm; and
a wavelength restriction unit for restricting a wavelength such that image capturing light employed for image capturing by the image capturing means for receiving reflection light or transmission light in the light irradiated at a subject exceeds the wavelength of 1200 nm, and becomes to have only a predetermined wavelength band including a wavelength band where the light transmittance of a predetermined living body tissue is equal to or greater than the light transmittance of the tube wall of a blood vessel or blood.

24. The infrared observation system according to claim 23, wherein the predetermined living body tissue is fat.

25. The infrared observation system according to claim 23, wherein the predetermined wavelength band is a wavelength band including at least a band of 1200 nm through 1600 nm, and a band of 1850 nm through 2200 nm.

26. The infrared observation system according to claim 23, wherein the predetermined wavelength band is a wavelength band including at least a band of 1200 nm through 2500 nm.

27. An infrared observation system comprising:
a light source unit for generating light of an infrared region having a long wavelength exceeding at least a wavelength of 1200 nm;
an infrared image capturing unit having sensitivity in an infrared region exceeding the wavelength of 1200 nm; and
a wavelength restriction unit for restricting a wavelength such that image capturing light employed for image capturing by the image capturing means for receiving reflection light or transmission light in the light irradiated at a subject exceeds the wavelength of 1200 nm, and becomes to have only a predetermined wavelength band including a wavelength where the difference between the light transmittance of a predetermined living body tissue and the tube wall of a blood vessel, and the transmittance of blood becomes the maximum.

28. The infrared observation system according to claim 27, wherein the predetermined living body tissue is fat.

29. The infrared observation system according to claim 27, wherein the predetermined wavelength band includes at least one band, of respective bands of 1650±50 nm, 1850±50 nm, and 2200±50 nm.

30. The infrared observation system according to claim 1, further comprising an irradiation unit for irradiating the infrared region light toward a living body tissue, and the irradiation unit is formed in treatment equipment which can be disposed at a position substantially facing the infrared image capturing unit sandwiching the irradiated living body tissue.

31. The infrared observation system according to claim 20, further comprising an irradiation unit for irradiating the infrared region light toward a living body tissue as the subject, and the irradiation unit is formed in treatment equipment which can be disposed at a position substantially facing the infrared image capturing unit sandwiching the irradiated living body tissue.

32. The infrared observation system according to claim 23, further comprising an irradiation unit for irradiating the infrared region light toward a living body tissue as the subject, and the irradiation unit is formed in treatment equipment which can be disposed at a position substantially facing the infrared image capturing unit sandwiching the irradiated living body tissue.

33. The infrared observation system according to claim 27, further comprising an irradiation unit for irradiating the infrared region light toward a living body tissue as the subject, and the irradiation unit is formed in treatment equipment which can be disposed at a position substantially facing the infrared image capturing unit sandwiching the irradiated living body tissue.

34. The infrared observation system according to claim 30, wherein the treatment equipment comprises a stock portion, and a surface portion including the irradiation unit, and the surface portion is provided at the end portion of the stock portion.

35. The infrared observation system according to claim 31, wherein the treatment equipment comprises a stock portion, and a surface portion including the irradiation unit, and the surface portion is provided at the end portion of the stock portion.

36. The infrared observation system according to claim 32, wherein the treatment equipment comprises a stock portion, and a surface portion including the irradiation unit, and the surface portion is provided at the end portion of the stock portion.

37. The infrared observation system according to claim 33, wherein the treatment equipment comprises a stock portion, and a surface portion including the irradiation unit, and the surface portion is provided at the end portion of the stock portion.

38. The infrared observation system according to claim 30, wherein the irradiation unit includes a single or multiple emission elements.

39. The infrared observation system according to claim 31, wherein the irradiation unit includes a single or multiple emission elements.

40. The infrared observation system according to claim 32, wherein the irradiation unit includes a single or multiple emission elements.

41. The infrared observation system according to claim 33, wherein the irradiation unit includes a single or multiple emission elements.

42. An infrared observation system comprising:
an image capturing unit for capturing the image of a subject, and outputting this as an image capturing signal;
an image processing unit for generating a picture signal for displaying the image of a subject on a display unit based on the image capturing signal, and outputting the picture signal to the display unit; and
treatment equipment including a irradiation unit for irradiating infrared light upon the subject, enabling the irradiation unit to be disposed at a position substantially facing the image capturing unit sandwiching the subject, and enabling the image capturing unit to capture the image of the subject using the transmission light of the infrared light irradiated upon the subject from the irradiation unit.

43. The infrared observation system according to claim 42, wherein the treatment equipment comprises a stock portion, and a surface portion including the irradiation unit, and the surface portion is provided at the end portion of the stock portion.

44. The infrared observation system according to claim 42, wherein the irradiation unit includes a single or multiple emission elements.

45. The infrared observation system according to claim 43, the treatment equipment further comprising:
multiple surface members constituting the surface portion, each of which includes the irradiation unit;
a shaft member for attaching each of the multiple surface members to the stock portion; and
treatment equipment operating-unit for moving each of the multiple surface members rotationally in a predetermined direction with the shaft member as a central shaft.

46. The infrared observation system according to claim 43, further comprising an endoscope provided in the tip portion of the image capturing unit, wherein the treatment equipment includes a curved portion which can change the emission direction of the infrared light to be emitted from the irradiation unit, and can be inserted into a duct provided in the inside of the endoscope.

47. The infrared observation system according to claim 42, wherein the wavelength band of the infrared light emitted by the irradiation unit includes at least the maximum absorption wavelength where the photo-absorption properties of oxygenated hemoglobin becomes the maximum.

48. The infrared observation system according to claim 42, wherein the wavelength band of the infrared light to be emitted by the irradiation unit includes at least the maximum absorption wavelength where the photo-absorption properties of hemoglobin becomes the maximum.
